(19) United States
(12) Patent Application Publication
(10) Pub. No.: US 2003/0073097 A1
(43) Pub. Date: Apr. 17, 2003

(54) TRAF6-regulated IKK activators (TRIKA1 and TRIKA2) and their use as anti-inflammatory targets

(76) Inventors: Zhijian J. Chen, Dallas, TX (US); Li Deng, Dallas, TX (US)

Correspondence Address: Steven L. Highlander, Fulbright & Jaworski L.L.P., Suite 2400, 600 Congress Avenue, Austin, TX, 78701, US

(21) Appl. No.: 10/076,918

(22) Filed: Oct. 11, 2001

(51) Int. Cl. C12Q 1/68; G01N 33/53; G01N 33/537; G01N 33/543; C12Q 1/42

(52) U.S. Cl. 435/6; 435/7.92; 435/21

(57) ABSTRACT

Proteins in the IKK and JNK signaling pathways, such as NFκB, are involved in the regulation of inflammatory diseases. Through phosphorylation and polyubiquitination, IκB proteins which sequester NFκB in the cytoplasm, are degraded by the ubiquitin-proteasome pathway releasing NFκB to the nucleus where it is activated. The present invention provides methods utilizing the composition of proteins in the IKK, JNK and ubiquitin-proteasome pathways such as, TRAF6 or TRAF2 (E3-ubiquitin protein ligase), TRIKA1/Uev1A/Ubc13 complex (E2-ubiquitin conjugating enzyme), and TRIKA2/TAK1 (protein kinase), in screening for candidate modulators involved in activation of the IKK and JNK pathways. The application further provides methods of utilizing the candidate modulators as drug therapeutics against inflammatory and immune diseases.

The government owns rights in the present invention pursuant to a grant from the National Institutes of Health.

### BACKGROUND OF THE INVENTION

1. Field of the Invention

The present invention relates generally to the fields of molecular biology biochemistry and immunology. More particularly, it concerns the mechanism of activation of the IKK and JNK signaling pathways in inflammatory and immune responses, and the targeting of these pathways in therapeutic modalities.

2. Description of Related Art

Although signal transduction pathways and many related cellular factors are fairly well delineated, the pathways responsible for the expression of many critical responses, such as inflammatory and immune responses, have not been completely elucidated.

Inflammatory diseases represent a large and increasing health burden throughout the world. Inflammatory diseases include autoimmune disorders such as rheumatoid arthritis, multiple sclerosis, psoriasis, asthma, cancers, heart diseases, diabetes, and viral infections. It has been suggested that the regulation of one or a few proteins in the related signaling pathways is a major etiologic factor in many of these diseases. Moreover, a variety of transcriptional regulatory factors such as transcription activators and transcription inhibitors are involved in the expression of these proteins. One of such transcriptional regulatory factors is NFκB.

NFκB provides a paradigm for a transcription factor that is regulated primarily via nuclear translocation (Sen and Baltimore, 1986; Baeuerle and Baltimore, 1988). NFκB plays a critical role in regulating the expression of a number of genes including cytokines, chemokines and other mediators of inflammatory and immune responses. NFκB is a dimeric transcription factor composed of p50 (NFκB1) and p60 (Re1A) subunits (Hatada et al., 2000). In unstimulated cells NFκB is bound to one of three IκB inhibitory proteins (IκBα, IκBβ, or IκBε) which is responsible for sequestering the NFκB/IκB complex in the cytoplasm. Many of the signals that lead to the activation of NFκB converge on IKK kinase, which consists of the catalytic subunits IKKα, IKKβ, and the regulatory subunit Nemo (also known as IKKγ).

Upon stimulation by agonists, such as tumor necrosis factor α (TNFα) and interleukin 1β (IL-1β), IκB kinase (IKK) complex is activated and subsequently rapidly phosphorylates IκB proteins. IκB kinase complex can also be independently activated by the mitogen-activated protein kinase kinase kinase family MAP3K; such as the extracellular signal-regulated kinase kinase kinase 1 MEKK1, a kinase of the JNK/SAPK pathway; and NFκB inducing kinase NIK.

Phosphorylation of IκB proteins leads to polyubiquitination of these proteins and their subsequent degradation by the 26S proteosome followed by release of NFκB. (Ghosh et al, 1998). The ubiquitin-proteasome pathway plays a major role in the selective degradation of proteins (Finley et al., 1991). It is further instrumental in a variety of cellular functions such as DNA repair, cell cycle progression, signal transduction, transcription, and antigen presentation. The ubiquitin-proteasome pathway is also regarded as playing a critical role in protein breakdown in inflammation and sepsis as well as in the regulation of inflammatory cell responses.

Despite the discovery of numerous new drugs over the last few decades, there is still a need for new, more efficient and less toxic compounds to combat diseases that elicit inflammatory and immune responses. The ability to identify such compounds is of vital importance and can be better addressed by elucidating the mechanism(s) involved in promoting inflammatory and immune responses, first by identifying then by targeting specific proteins involved in the pathways such as the IKK, JNK, and ubiquitin-proteasome pathways

### SUMMARY OF THE INVENTION

The present invention overcomes the deficiencies in the art by identifying proteins that are specifically involved in the IKK, JNK and ubiquitin-proteasome pathways that are further involved in eliciting inflammatory and immune responses. This information is exploited in the development of drug screening and therapeutic applications.

Thus, in accordance with the present invention there is provided a method of screening for modulators of IKK and JNK activation comprising (a) providing a Ubc13/Uev1A complex and TRAF6 or TRAF2; (b) contacting the complex and TRAF6 or TRAF2 with a candidate modulator substance in the presence of E1, a plurality of ubiquitin molecules and ATP; (c) determining the formation of free polyubiquitin chains, wherein a change in poly-ubiquitin formation in the presence of the candidate modulator, as compared with poly-ubiquitin formation in the absence of the candidate modulator, indicates that the candidate modulator is an modulator of IKK and JNK activation.

The method may further comprise measuring poly-ubiquitin formation in the absence of the candidate modulator. The candidate modulator may be a protein or peptide, an expression construct, or an organic or inorganic small molecule, a DNA oligonucleotide or its analogue or an RNA oligonucleotide or its analogue. It may be an inhibitor or a stimulator. The method may further comprise measuring poly-ubiquitin formation by immunodetection. The immunodetection may comprise detecting ubiquitin fused to an immunodetectable marker, such as myc or His6X. The format of the immunodetection may be ELISA. The plurality of ubiquitin molecules may comprise a single lysine residue at position 63.

In another embodiment, there is provided a method of screening for modulators of IKK and JNK activation comprising (a) providing a Ubc13/Uev1A complex, TRAF6 or TRAF2; (b) contacting TAB1/TAB2/TAK1 complex, Ubc13/Uev1A complex, TRAF6 or TRAF2 with a candidate modulator substance in the presence of E1, a plurality of ubiquitin molecules and ATP; (c) determining the polyubiquitination of TRAF6 or TRAF2, wherein a change in the phosphorylation state of IKK or MKK in the presence of the candidate modulator, as compared with the phosphorylation state of IKK or MKK in the absence of the candidate modulator, indicates that the candidate modulator is an modulator of IKK and JNK activation.

The method may further comprise measuring the polyubiquitination state of TRAF6 or TRAF2 in the absence of the candidate modulator. The candidate modulator a protein or peptide, an expression construct, an organic or inorganic small molecule, a DNA oligonucleotide or its analogue, or an RNA oligonucleotide or its analogue. The candidate modulator may be an inhibitor or a stimulator. The method may further comprise measuring poly-ubiquitin state of TRAF6 or TRAF2 by immunodetection. The immunodetection may comprise detecting TRAF2, TRAF6 or ubiquitin fused to an immunodetectable marker. The ubiquitin molecule may comprise a single lysine residue at position 63. The immunodetectable marker may be 6HisX or myc. The format of the immunodetection may be ELISA.

In yet another embodiment, there is provided a method of screening for modulators of IKK and JNK activation comprising (a) providing a TRAF2 or a TRAF6; (b) contacting TRAF2 or TRAF6 with a candidate modulator substance in the presence of E1, Ubc13/Uev1A, a plurality of ubiquitin molecules and ATP; (c) determining the ubiquitin ligase activity of TRAF2 or TRAF6, wherein a change in the enzyme activity of TRAF2 or TRAF6 in the presence of the candidate modulator, as compared with the activity of TRAF2 or TRAF6 in the absence of the candidate modulator, indicates that the candidate modulator is an modulator of IKK and JNK activation.

The method may further comprise measuring TRAF2 or TRAF6 activity in the absence of the candidate modulator. The candidate modulator may be a protein or peptide, an expression construct, or an organic or inorganic small molecule, a DNA oligonucleotide or its analogue or an RNA oligonucleotide or its analogue. It may be an inhibitor or a stimulator. The assay may further comprise measuring poly-ubiquitin formation by immunodetection, for example, where immunodetection comprises detecting ubiquitin fused to an immunodetectable marker. The immunodetectable marker may be myc or His6X.

In still a further embodiment, there is provided a method of screening for modulators of IKK and JNK activation comprising (a) providing TAK1, TAB1, TRAF6 and TAB2; (b) contacting TAK1, TAB1, TRAF6 and TAB2 with a candidate modulator substance in the presence of E1, Ubc13/Uev1A, a plurality of ubiquitin molecules and ATP; (c) determining the kinase activity of TAK1,wherein a change in the kinase activity of TAK1 in the presence of the candidate modulator, as compared with the kinase activity of TAK1 in the absence of the candidate modulator, indicates that the candidate modulator is an modulator of IKK and JNK activation.

The method may further comprise measuring TAK1 activity in the absence of the candidate modulator. The candidate modulator may be a protein or peptide, an expression construct, or an organic or inorganic small molecule, a DNA oligonucleotide or its analogue or an RNA oligonucleotide or its analogue. It may be an inhibitor or a stimulator. TAK1 kinase activity may be measured by determining the phosophorylation state of IκBα incubated with the TAK1, for example, where the phosphorylation state is determined with an antibody that binds selectively to phosphorylated IκBα. The format of the assay may be an ELISA.

In still yet a further embodiment, there is provided a method of screening for modulators of IKK and JNK activation comprising (a) providing TAK1, TAB1, TAB2, TRAF6 and IKK complex; (b) contacting TAK1, TAB1, TAB2, TRAF6 and IKK complex with a candidate modulator substance in the presence of E1, Ubc13/Uev1A, a plurality of ubiquitin molecules and ATP; (c) determining the phosphorylation state of IκB and MKK, wherein a change in the phosphorylation state of IκB and MKK in the presence of the candidate modulator, as compared with the phosphorylation state of IκB and MKK in the absence of the candidate modulator, indicates that the candidate modulator is an modulator of IKK and JNK activation.

The method may further comprise measuring the phosphorylation of IκBα or MKK in the absence of the candidate modulator. The candidate modulator may be a protein or peptide, an expression construct, or an organic or inorganic small molecule, a DNA oligonucleotide or its analogue or an RNA oligonucleotide or its analogue. It may be an inhibitor or a stimulator. The method phosphorylation state of IκBα or MKK may be determined with an antibody that binds selectively to phosphorylated IκBα or MKK. The format of the assay may be an ELISA.

Other embodiments of the invention, as well as a more detailed discussion of the inventions described above, are provided in the following pages.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: Purification and identification of TRIKA1. FIG. 1A Purification scheme. FIG. 1B Amino acid sequence of human Ubc13. FIG. 1C Amino acid sequence of human Uev1A. Underlined are the peptides whose masses match those of TRIKA1α and TRIKA1β, respectively.

FIG. 2: TRIKA1 is required for IKK activation by TRAF6. Interference of Ubc13/Uev1A inhibits NF-κB activation in cells. The expression construct encoding Ubc13(C87A) was cotransfected into 293 cells together with a luciferase reporter gene (κB₃-Luc or Gal4-Luc). NF-κB was induced by stimulation with IL-1β (20 ng/ml) or TNFα(20 ng/ml) for 6 hours, or by cotransfection with expression constructs encoding TRAF6 (300 ng DNA), TRAF2 (200 ng), NIK (10 ng), HTLV1 TAX (20 ng), Gal4-Re1 (300 ng) or Gal4-VP16 (100 ng). The luciferase activity was measured 48 hours after transfection. The result shown was an average of duplicated experiments.

FIG. 3: TRAF6 facilitates the assembly of K63-linked polyUb chains in conjunction with Ubc13/Uev1A. An intact RING finger is required for NF-κB activation by TRAF6. Expression plasmids encoding TRAF6 and mutants were transfected into 293 cells together with the reporter κB₃-Luc. 48 hours after transfection, cells were harvested for luciferase activity assays.

FIG. 4: K63-linked polyUb chains mediate the activation of IKK in response to TRAF6. Illustration of Ub lysine mutants.

FIG. 5: TAK1 is a ubiquitin-dependent kinase of the MKK/JNK pathway. Immunopurified TAK1 complex was subjected to ubiquitination in the presence of Ubc13/Uev1A and TRAF6 together with various Ub mutants, and then assayed for phosphorylation of MKK6.

FIG. 6: K63-linked polyubiquitination of TRAF6. Diagrams of TRAF6-GyraseB chimeric constructs.

### DESCRIPTION OF ILLUSTRATIVE EMBODIMENTS

Though much has been learned regarding signalling pathways, the precise mechanisms involving activation of a number of key biological responses remain obscure. Of particular importance are inflammatory reactions, which are triggered by a number of different stimuli, and which manifest themselves in a wide variety of pathologic states. Both the IKK and JNK pathways are central to these clinical situations. Thus, there remains a need to improve the understanding of these pathways, and to seek ways to exploit them from a therapeutic standpoint.

I. The Present Invention

The present invention, by elucidating signaling mechanisms involved in eliciting inflammatory and immune responses to a disease, provides methods for intervening in the ubiquitin, JNK and IKK signaling pathways. In particular, the present invention seeks to specifically target molecules in these pathways with potent inhibitors, thereby providing new methods for dealing with the associated disease.

Thus, in one embodiment of the present invention, the present inventors exploit the discovery that TRAF6 and TRAF2 proteins function as a ubiquitin ligases. In another embodiment, the invention involves use of the ubiquitin conjugating enzymes Ubc13 and UevA1 as part of a functional complex (TRIKA1) in activation of the IKK pathway. In yet another embodiment, TRIKA2, which comprises TAK1, TAB1 and TAB2 (TRIKA2/TAK1 complex), is identified as a new class of protein kinases whose activity is regulated by ubiquitination, thereby identifying a novel regulatory mechanism that is exploited in the present invention. In other embodiments, the JNK pathway also is regulated by TRIKA1 and TRIKA2 proteins, demonstrating cross-talk between the signaling pathways that can be exploited.

Thus, one of the major advantages of the present invention lies in the ability to target particular proteins involved in the NFκB pathway, and subsequently the IKK, JNK and ubiquitin-proteasome pathways, in treating inflammatory diseases. By identifying inhibitors of these pathways, and in particular, of the specifically identified interactions, the benefits of precise inhibition of enzymatic activity can be realized.

II. The IKK and JNK Pathways

NFκB plays a critical role in regulating the expression of a number of genes including cytokines, chemokines and other mediators of inflammatory and immune responses. NFκB/Re1 transcription factor family consist of five mammalian family members which bind DNA as homodimers or heterodimers. NFκB is a dimeric transcription factor composed of p50 (NFκB1) and p60 (Re1A) subunits (Hatada et al., 2000) and is primarily regulated via nuclear translocation. In unstimulated cells NFκB is bound to one of three IκB inhibitory proteins (IκBα, IκBβ, or IκBε) which is responsible for sequestering the NFκB/IκB complex in the cytoplasm. IκBs retain NFκB in the cytoplasm by masking its nuclear localization signal. Many of the signals that lead to the activation of NFκB converge on IKK kinase which consists of the catalytic subunits IKKBα, IKKβ, and the regulatory subunit Nemo (also known as IKKBγ).

Upon stimulation by agonists, such as tumor necrosis factor a (TNFα) and interleukin 1β (IL-1β), IκB kinase (IKK) complex is activated and subsequently rapidly phosphorylates IκB proteins. This phosphorylation event is specific in that it occurs only on serine residues; Ser32 and 36 on IκBα and Ser 19 and 23 on IκBβ. IκB kinase complex can also be independently activated by the mitogen-activated protein kinase kinase kinase family MAP3K; such as the extracellular signal-regulated kinase kinase kinase 1 MEKK1, a kinase of the JNK/SAPK pathway; and NFκB inducing kinase NIK. Phosphorylation of IκB proteins leads to polyubiquitination and subsequent degradation of these proteins followed by the release of NFκB. (Ghosh et al, 1998).

Ubiquitination/polyubiquitination of proteins occurs through the ubiquitin-proteasome pathway which plays a major role in the selective degradation of proteins (Finley et al., 1991); and is further instrumental in a variety of cellular functions such as DNA repair, cell cycle progression, signal transduction, transcription, and antigen presentation. The ubiquitin-proteasome pathway is also regarded as playing a critical role in protein breakdown in inflammation and sepsis as well as in the regulation of inflammatory cell responses.

The ubiquitin protein is a small protein of about 76 amino acids that is highly conserved, and has many diverse functions such as the degradation of proteins. Ubiquitination is the covalent modification of various cellular proteins. Degradation of proteins by the ubiquitin-proteasome pathway involves conjugation of multiple molecules of ubiquitin to the target protein, and the degradation of the tagged substrate by the 26S proteasome. Conjugation of one or more ubiquitin molecules to a target substrate is a three step process. This reaction is initated by the ubiquitin activating enzyme E1 which activates ubiquitin in its C-terminal glycine by adenylation, to form a high-energy intermediate. Thereafter, a second reaction step is involved in which ubiquitin is transferred to a thiol site and becomes linked to E1 via a thiolester bond. This reaction step is requires ATP. Following activation by E1, ubiquitin is transferred to a ubiquitin conjugating enzymes (E2) generating another thioester linkage. At this point ubiquitin can either be linked directly to a target protein or conjugated through E3 (ubiquitin protein ligases). E2 can directly attach ubiquitin to a lysine residue in a target protein but most often the reaction will require E3—the ubiquitin ligase that has been implicated in the recognition and transfer of ubiquitin from E2 to E3 via the thiolester intermediate.

Conjugated ubiquitin can itself be further ubiquitinated through one of its seven lysine residues to form a polyubiquitin chain. The seven lysine residues that have been shown to be involved in polyubiquitination are located at posistion 6, 11, 27, 29, 33, 48 and 63. Lysine 63-linked polyubiquitination chains have been shown to play a critical role in TRAF6 mediated IKK activation (Deng et al., 2000). A typical ubiquitin to ubiquitin linkage within a poylubiquitin chain recognizable by the proteasome, involves lysine at position 48 of one ubiquitin molecule and the carboxyl terminus of the other ubiquitin molecule (Chan et al, 1989). Ubiquitin activated by E1 and transferred to E2 which in conjunction with E3 conjugates ubiquitin onto a lysine residue of the target protein forms an isopeptide bond (Laney & Hochstrasser, 1999). Although E2 can directly attach ubiquitin to a lysine residue in the substrate, this reaction is most often requires the ubiquitin-protein ligase E3 to which the target protein is specifically bound. However, several proteins are targeted by specific pairs of E2 and E3 enzymes which recognize distnct structural motifs.

The final step in the ubiquitin conjugaton process involves the covalent attachment of ubiquitin to the substrate followed by the generation of a polyubqutin chain anchored to an ε-NH₂ group of a lysine residue in the target protein. Additionally certain amino acid sequences may be signaled for degradation such as PEST (proline, glutamic acid, serine and threonine) sequences. The binding of a target protein to E3 is specific and implies that E3 plays a major role in the recognition and selection of proteins for conjugation by the ubiquitin-proteasome pathway. The target protein tagged by the conjugation of polyubiquitin chains, is subsequently recognized and degraded by the 26S proteasome.

Further crosstalk between the signaling pathways is demonstrated by TNFα and IL-1, the most potent activators of NFκB. Although TNF and IL-1 initiate signaling cascades leading to NFκB activation via distinct families of cell-surface receptors (Smith et al., 1994; Dinarello, 1996), both pathways utilize members of the TNF receptor-associated factor (TRAF) family of adaptor proteins as signal transducers (Rothe et al., 1995; Hsu et al., 1996; Cao et al., 1996b). TRAF proteins were originally found to associate directly with the cytoplasmic domains of several members of the TNF receptor family including the 75 kDa TNF receptor (TNFR2), CD40, CD30, and the lymphotoxin-beta. receptor (Rothe et al., 1994; Hu et al., 1994; Cheng et al., 1995; Mosialos et al., 1995; Song and Donner, 1995; Sato et al., 1995; Lee et al., 1996; Gedrich et al., 1996; Ansieau et al., 1996). In addition, TRAF proteins are recruited indirectly to the 55 kDa TNF receptor (TNFR1) by the adaptor protein TRADD (Hsu et al., 1996). Activation of NFκB by TNF requires TRAF2 (Rothe et al., 1995; Hsu et al., 1996). TRAF5 has also been implicated in NFκB activation by members of the TNF receptor family (Nakano et al., 1996). In contrast, TRAF6 participates in NFκB activation by IL-1 (Cao et al., 1996b). Upon IL-1 treatment, TRAF6 associates with IRAK, a serine-threonine kinase that binds to the IL-1 receptor complex (Cao et al., 1996a; Huang, 1997). TRAF proteins also interact with NIK and thereby most likely activate NIK. TNFα and IL-1 also activate other related signaling pathways and transcription factors through activation of JNK(c-Jun N-terminal kinase). Signal-induced activation of JNK may diverge from NFκB activation at the level of a group of adapter proteins, the TNF receptor associated factor proteins (TRAFs). Activated TRAFs in turn activate the JNK pathway through an unknown mechanism.

In light of the information provided herein, one of skill in the art can now explore the IKK and JNK pathways with a complete understanding of how they operate. This provides three notable advantages. First, one can construct an entire signalling pathway artificially, thereby controlling the function of each element. Second, one can also focus in on the interactions of particular parts of the pathway, thereby developing agents that are unique in their location of attack. And third, one can generate new reagents that permit, in a much more facile way, the screening of agents for these pathways.

III. Screening for Modulators of the IKK and JNK Pathways

Thus, in accordance with the present invention, there are provided methods for identifying modulators of the function of various members of the IKK and JNK pathways. Such methods involve assays for candidate substances that modulate phosphorylation, polyubiquitination, ligase and kinase activity of various molecules. These assays may comprise random screening of large libraries of candidate substances; alternatively, the assays may focus on particular classes of compounds selected with an eye towards structural attributes that are believed to make them more likely to modulate the function and expression of the targeted proteins or interactions.

To identify a modulator, one generally will determine the function or activity of the target protein in the presence and absence of the candidate substance, a modulator defined as any substance that alters function or expression. Assays may be conducted in cell free systems, or in isolated cells.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance expression or activity of the target protein. The candidate substance may be a protein or peptide, an expression construct, an organic or inorganic small molecule, an inhibitor or stimulator, a DNA or RNA oligonucleotide or its analogue. It may prove to be the case that the most useful pharmacological compounds will be compounds that are structurally related to the target, its cofactors or any other interacting molecules. Using lead compounds to help develop improved compounds is known as “rational drug design” and includes not only comparisons with known inhibitors and activators, but predictions relating to the structure of target molecules.

The goal of rational drug design is to produce structural analogs of biologically active polypeptides or target compounds. By creating such analogs, it is possible to synthesize drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be a protein or peptide, an expression construct, an organic or inorganic small molecule, an inhibitor or stimulator, a DNA or RNA oligonucleotide or its analogue, or any other compounds that may be designed through a rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on protein targets. Regardless of the type of inhibitor or activator identified by the present screening methods, the effect of the inhibition or activator by such a compound results in modulation as compared to that observed in the absence of the added candidate substance.

2. In vitro Assays

To more directly analyze and further elucidate the interactions of proteins or compounds of interest, a cell free system is ideal in that it allows the establishment of the mechanism without the complexity of all the biological interactions that would be present in a cell. Moreover, a cell free system allows for manipulation of the various components to be tested. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding. Various examples of binding assays in accordance with the present invention would include assays to measure binding of TAK1 and TAB1, TAB1 and TAB2, TAK1 and TAB2, TAK1/TAB1 and TAB2, TRAF6 or TRAF2 and TRIKA1, TRIKA2 and IκB or MKK, and Ubc13 and Uev1A.

A technique for high throughput screening of compounds is described in WO 84/03564. Large numbers of small peptide test compounds are synthesized on a solid substrate, such as plastic pins or some other surface. Bound polypeptide is detected by various methods. More specifically, examples of high throughput screening assays that may be employed in the present invention are discussed herein.

3. Specific Assays

(i) Polyubiquitination Assays

Ubiquitin is conjugated on the lysine residue of the target protein to form a isopeptide bond. Polyubiquitination of conjugated ubiquitin further occurs through one of its seven lysines, such as lysine at position 63, to form a polyubiquitin chain. The present invention therefore seeks to determine the formation of free polyubiquitination chains in the presence and absence of a candidate substance. Thus, in one embodiment, a cell free system is provided comprising the ubiquitin conjugating enzyme complex Ubc13/Uev1A (TRIKA1) which is involved in the formation of the ubiquitin-conjugate enzyme (E2). Along with TRAF proteins such as TRAF6 or TRAF2, which serve as ubiquitin ligases (E3), E1, a ubiquitin activating enzyme, and ATP, a functional systems is provided. Quenching of the reaction is achieved by adding 10-20 mM EDTA. Of particular interest is the use of a ubiquitin mutant that contains a single lysine at residue 63, thereby limiting the formation of polyubiqutin to this residue.

Immunodetection of free polyubiquitin chains provides the “read out” for the assay, in a particular embodiment, using a myc- or His6X-tagged ubiquitin molecule for capture and subsequent detection. Nickel coating of an appropriate surface also permits first stage capture of ubiquitin (nickel is a charged compound which facilitates the binding of His6X). The polyubiquitination of TRAF6 or TRAF2 can also be measured in a similar fashion, including use of a myc- or His6X-tagged TRAF molecule, although the additional inclusion of TRIKA2 (TAB 1, TAB2 and TAK1) is required.

(ii) Phosphorylation Assays

TAK1 is a kinase, which is an enzyme that transfers a phosphate group to a target molecule. In measuring the activity of TAK1, a variety of different components are required. In addition to TAK1, a reaction mixture contains E1, Ubc13/Uev1A. TRAF6, ubiquitin, TAK1/TAB1/TAB2 (TRIKA2), IKK complex, IκBα and ATP. In this reaction, IκBα is the target; it may be fused to a protein such as GST for isolation purposes. After incubation in the presence or absence of inhibitors, the reactions can be quenched by adding 10-20 mM EDTA.

To detect phosphorylated IκBα, reaction mixture is treated such that the IκBα is isolated, for example, on a microtitre plate coated with glutathione to capture GST-IκBα. Phosphorylation of IκBα can then be measured using an antibody specific for phospho-IκBα. A similar assay looking at MKK phosphorylation may be employed.

4. Selectable and Screenable Markers

In accordance with the present invention, it may prove useful to generate recombinant cells that contain reporter gene constructs, especially in the secondary testing of candidate drugs initially identified in cell free assays. These constructs would comprise, generally, a promoter derived from a gene that is regulated by the IKK and/or JNK pathways. Under the control of this promoter would be a screenable or selectable marker. Thus, one could quickly determine, in the context of the recombinant cells, whether signalling through the relevant pathway(s) had been altered by the provision of a candidate substance.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed in the host cell of interest. Further examples of selectable and screenable markers are well known to one of skill in the art.

IV. Immunodetection Methods

Biological interactions, such as polyubiquitin formation, the ubiquitination of TRAF6 or TRAF2, or the phosphorylation of IKK and MKK by the kinase activity of TRIKA2/TAK1 complex, can be examined by immunodetection methods, as discussed briefly above. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature (e.g., Doolittle & Ben-Zeev, 1999; Gulbis & Galand, 1993; and De Jager et al., 1993).

In general, the immunobinding methods include contacting a sample with an antibody or fragment thereof, under conditions effective to allow the formation of immunocomplexes. The antibody serves to isolate the protein from other components in the sample so that it can be assayed. The antibody may be linked to a solid support, such as in the form of a column matrix. The unwanted components will be washed from the column, leaving the antigen immunocomplexed to the immobilized antibody to be eluted.

Contacting the biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, an ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls.

1. ELISAs

The present invention utilizes ELISAs as known to the skilled artisan to detect and quantify the formation of polyubiquitination chains, polyubiquitination of TRAF6 or TRAF2, and phosphorylation of IκBα and MKK in the presence and absence of a candidate substance as described herein.

As detailed above, immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound protein, polypeptide or peptide antigen may be detected. Detection is generally achieved by the addition of another antibody that is linked to a detectable label. This type of ELISA is a simple “sandwich ELISA.”Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the antibodies of the invention. After binding and washing to remove non-specifically bound immune complexes, the bound antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the proteins, polypeptides or peptides are immobilized, involves the use of antibody competition in the detection. In this ELISA, labeled antibodies against a protein, polypeptide or peptides are added to the wells, allowed to bind, and detected by means of their label. The amount of antigen in an unknown sample is then determined by mixing the sample with the labeled antibodies against the protein before or during incubation with coated wells. The presence of a proteinaceous molecule in the sample acts to reduce the amount of antibody against a proteinaceous molecule available for binding to the well and thus reduces the ultimate signal. This is also appropriate for detecting antibodies against a protein, polypeptide or peptide in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a proteinaceous molecule or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hrs or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation (e.g., incubation for 2 hrs at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid (ABTS) and H₂O₂, in the case of peroxidase as the enzyme label. Quantification is then achieved by measuring the degree of color generation, e.g., using a visible spectra spectrophotometer.

2. Immunoblot or Western Blot

The compositions of the present invention may find use in immunoblot or western blot analysis. The antibodies of the present invention may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. This is especially useful when the antigens studied are immunoglobulins (precluding the use of immunoglobulins binding bacterial cell wall components), the antigens studied cross-react with the detecting agent, or they migrate at the same relative molecular weight as a cross-reacting signal.

Immunologically-based detection methods for use in conjunction with Western blotting include enzymatically-, radiolabel-, or fluorescently-tagged secondary antibodies against the protein moiety are considered to be of particular use in this regard.

3. Epitope Tagging

Detection of polyubiquitin chain formation, polyubiquitination of TRAF6 or TRAF2, and phosphorylation of IκB by immunoassays, such as ELISAs and Western blotting, may incorporate proteins that are labeled or tagged. Epitope tagging is a technique widely employed for detecting proteins, and may be particularly useful for discriminating among similar proteins or peptides that are not distinguishable with conventional antibodies. Epitope tagging involves adding a unique epitope tag peptide sequence to the protein of interest by recombinant DNA techniques, thereby creating a fusion protein. The resulting tagged protein can then be detected with an antibody specific for the epitope tag.

Epitope tagging of a protein employs conventional means as known to the skilled artisan. This process involves two DNA molecules: (1) a polynucleotide which is cloned in a plasmid vector and which includes a sequence of nucleotides encoding the protein as well as regulatory sequences (i.e. promoter, translations start, etc.) needed to express the protein; and (2) an oligonucleotide encoding the epitope with which the protein is to be tagged. The oligonucleotide is designed to encode, in one of its reading frames, an epitope recognized by a known antibody. A site in the polynucleotide protein coding sequence for insertion of the oligonucleotide is designated. This site may be located at or near the 3′ or the 5′end of the coding sequence, or somewhere in between the 3′ and 5′ ends. The insertion site for the oligonucleotide is typically a unique restriction site. The plasmid is then linearized with the restriction endonuclease, and the oligonucleotide is ligated into the site.

The small size of the epitope tag, which is usually 5-20 amino acids in length, generally has no effect on the biological function of the tagged protein. This contrasts with many larger fusion proteins, in which the activity or function of the fusion protein is affected by longer peptide label. Using conventional epitope tagging techniques, as is known to the skilled artisan, hundreds of different proteins have been epitope-tagged with numerous distinct peptides. These including: the c-myc epitope (Evans et al., 1985); hexahistidine (His6) epitope (Caspers et al., 1991); Glutathione glutathione S-transferase (GST) epitope (Sato et al., 1983); the HA-epitope (Niman et al., 1983) the FLAG epitope (Hopp et al., 1988); the epsilon-tag epitope (Olah et al., 1994); the AU1 and AU5 epitopes (Lim et al., 1990); the glu-glu epitope (Grussenmeyer et al., 1985); the KT3 epitope (MacArthur et al., 1984); the IRS epitope (Liang et al., 1996); the BTag epitope (Wang et al., 1996); and the vesicular stomatitis virus (VSV) epitope (Kreis et al., 1986). Essentially any peptide can be used as an immunogen to raise antibodies that will recognize that same peptide when it is present within or at the termini of a protein.

The use of epitope tagging is advantageous in that it makes use of antibodies with known characteristics, and makes extensive characterization of new antibodies unnecessary. Epitope tagging is far less time consuming than the traditional method of producing an antibody to the specific protein being studied.

V. Nucleic Acid Delivery and Cell Transformation

In order to generate cells that express various members of the IKK and JNK pathways, such as members of the TRIKA1 and TRIKA2 complexes, as well as TRAF2, TRAK6, MKK, IκBα and ubiquitin, it may prove useful to transfer a nucleic acid encoding one of these molecules into a host cell. Such cell lines would express high levels of these proteins from which the proteins could be isolated (see below).

Suitable methods for nucleic acid delivery for transformation of an organelle, a cell, a tissue or an organism, for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); and any combination of such methods. Through the application of techniques such as these, organelle(s), cell(s), tissue(s) or organism(s) may be stably or transiently transformed.

1. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of inflammatory and immune response modulator used may vary upon the nature of the antigen as well as the organelle, cell, tissue or organism used

2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

5. Sonication Loading

Additional embodiments of the present invention include the introduction of a nucleic acid by direct sonic loading. LTK-fibroblasts have been transfected with the thymidine kinase gene by sonication loading (Fechheimer et al., 1987).

6. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet farther embodiments, a liposome may be complexed or employed in conjunction with both HVJ and HMG-1. In other embodiments, a delivery vehicle may comprise a ligand and a liposome.

7. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

VI. Protein Purification

In order to isolate the various components of the IKK and JNK pathways, primarily for using in cell free assays, one or more purification methods as known to the skilled artisan, may be further implemented by the present invention. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide. In a particular embodiment, recovery of proteins following recombinant production is envisioned.

Protein purification techniques are well known to those of skill in the art. These techniques involve, at one level, the crude fractionation of the cellular millieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition, that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

VII. Therapies

In accordance with the present invention, there also are provided methods for the treatment of various inflammatory diseases. These disease would be those triggered by the IKK and JNK pathways, and involving NF-κB. The primary causes of such inflammatory reactions are tumor necrosis factor and IL-1. Diseases that may be treated in accordance with the present invention include, but are not limited to, rheumatoid arthritis, asthma inflammatory bowel disease and psoriasis, allergic rhinitis, various dermatological conditions, acute pancreatitis; ALS; Alzheimer's disease; cachexia/anorexia; atherosclerosis; chronic fatigue syndrome, fever; diabetes (e.g., insulin diabetes); glomerulonephritis; graft versus host rejection; hemohorragic shock; hyperalgesia, inflammatory conditions of a joint, including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; congestive heart failure, cardiac hypertrophy; intraamniotic infection; reperfusion injury; septic shock; side effects from radiation therapy, temporal mandibular joint disease, tumor metastasis; or an inflammatory condition resulting from strain, sprain, cartilage damage, trauma, orthopedic surgery, bacterial and viral infections, bacterial meningitis sexually transmitted diseases; gonorrhea, chlamydia and trichomoniasis.

In addition to use of the inhibitors identified according to the present invention, one may also utilize inhibitors based on the nucleic acid sequence of particular enzymes in the IKK and JNK pathways. Such suitable targets include TRAF6, TRAF 2, and members of the TRIKA1 and TRIKA2 complexes. This may be accomplished using transgenes that are not expressed as proteins, i.e., transcribed but not translated. DNA may be introduced into organisms for the purpose of expressing RNA transcripts that function to affect phenotype yet are not translated into protein, or for direct inhibition of transcription or translation.

i. Antisense RNA

In certain aspects, a nucleic acid that is “antisense” to a target gene may be introduced into a cell. The nucleic acid may itself hybridize with and inhibit the target, or it may be expressed as an RNA, an “antisense message.” Nucleic acids may be constructed or, which when transcribed, produce antisense RNA that is complementary to all or part(s) of a targeted messenger DNA or RNA. The antisense molecule reduces production of the corresponding polypeptide product. The aforementioned genes will be referred to as antisense genes.

An antisense gene may thus be introduced into a cell by transformation methods to produce a novel transgenic cell or organism with reduced expression of a selected protein of interest. In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example of an antisense construct is a “peptide nucleic acid,” also known as a “PNA,” “peptide-based nucleic acid analog” or “PENAM,” described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit expression, as described in U.S. Pat. No. 5,891,625. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

ii. Ribozymes

In other aspects, the therapeutic transgene may produce a ribozyme. Nucleic acids may be constructed or isolated which, when transcribed, produce RNA enzymes (ribozymes) that can act as endoribonucleases and catalyze the cleavage of RNA molecules with selected sequences. The cleavage of selected messenger RNAs can result in the reduced production of their encoded polypeptide products. These genes may be used to prepare novel one or more cells, tissues and organisms which possess them. The transgenic cells, tissues or organisms may possess reduced levels of polypeptides including, but not limited to, the polypeptides cited above.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes.

Several different ribozyme motifs have been described with RNA cleavage activity (Symons, 1992). Examples include sequences from the Group I self splicing introns including Tobacco Ringspot Virus (Prody et al., 1986) and Lucerne Transient Streak Virus (Forster and Symons, 1987). Sequences from these and related viruses are referred to as hammerhead ribozyme based on a predicted folded secondary structure.

Other suitable ribozymes include sequences from RNase P with RNA cleavage activity (Yuan et al., 1992, Yuan and Altman, 1994, U.S. Pat. Nos. 5,168,053 and 5,624,824), hairpin ribozyme structures (Berzal-Herranz et al., 1992; Chowrira et al, 1993) and Hepatitis Delta virus based ribozymes (U.S. Pat. No. 5,625,047). The general design and optimization of ribozyme directed RNA cleavage activity has been discussed in detail (Haseloff and Gerlach, 1988, Symons, 1992, Chowrira et al., 1994; Thompson et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Perriman et al., 1992; Thompson et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in down regulating a given gene is simply a matter of preparing and testing a given sequence, and is a routinely practiced “screening” method known to those of skill in the art.

iii Combination Therapies

In order to enhance the effectiveness of an anti-inflammatory therapy, it may be desirable to combine the compositions and methods of the present invention with an agent effective in the treatment of inflammatory and immune response diseases. Thus, modulators that interact with TRIKA2/TAK1, TRIKA1, TRAF6 or TRAF2 proteins in the IKK and JNK signaling pathway can be used in combinations to more effectively combat an inflammatory or immune response. Furthermore, this process may involve contacting the cell(s) with an agent(s) and the target gene modulation at the same time or within a period of time wherein separate administration of the modulator and an agent to a cell, tissue or organism produces a desired therapeutic benefit. The terms “contacted” and “exposed,” when applied to a cell, tissue or organism, are used herein to describe the process by which a modulator and/or therapeutic agent are delivered to a target cell, tissue or organism or are placed in direct juxtaposition with the target cell, tissue or organism. The cell, tissue or organism may be contacted (e.g., by adminstration) with a single composition or pharmacological formulation that includes both a modulator and one or more agents, or by contacting the cell with two or more distinct compositions or formulations, wherein one composition includes a modulator and the other includes one or more agents.

The modulator of the present invention may precede, be co-current with or follow the other agent(s) by intervals ranging from minutes to weeks. In embodiments where the modulator and other agent(s) are applied separately to a cell, tissue or organism, one would generally ensure that a significant period of time did not expire between the time of each delivery, such that the modulator and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the modulator. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 12 hours, about 18 hours, about 24 hours, about 36 hours, about 48 hours, about 1 day, about 2 days, about 3 days, about 4 days, about 5 days, about 6 days, about 7 days, about 14 days, about 21 days, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, or about 12 months, and any range derivable therein, prior to and/or after administering the modulator.

Various combination regimens of the modulator and one or more agents may be employed. Non-limiting examples of such combinations are shown below, wherein a composition modulator is “A” and the other agent is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of modulators to a cell, tissue or organism may follow general protocols for the administration of inflammatory and immune therapeutics, taking into account the toxicity, if any. It is expected that the treatment cycles would be repeated as necessary. In particular embodiments, it is contemplated that various additional agents may be applied in any combination with the present invention. Suitable anti-inflammatory agents include the NSAIDs (aspirin, ibuprofen, naproxen, celecoxib, rofecoxib, sulindac, etc.), Advil, Aleve, Anaprox, Diclofenac, Docosahexaenoic acid, Dolobid, Etodolac, Feldene, Flurbiprofen, Indomethacin, Ketorolac tromethamine, Lodine, Meclofenamate, 6-MNA, Motrin, Nalfon, Naprosyn, Nuprin, Orudis, Phenylbutazone, Piroxicam, Phenylbutazone, Ponstel, Relafen, Tolectin, Toradol, Voltaren; also 5-lipoxygenase inhibitors, phosphodiesterase inhibitors, or cyclooxygenase inhibitors (e.g., cyclosalicylazosulfapyridine or azulfasalazine). Salicylic acid, salicylates, such as Asacol, Disalcid, Pentesa, Salflex, or Trilisate; corticosteroids (betamethasone, prednisone, methylprednisolone, etc.).

VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Cells and Antibodies. HeLa S3 cells were purchased from the Cell Culture Center (Minneapolis). The antibodies against IκBα (C21), Myc (9E10), and Ub (P4D1) were purchased from Santa Cruz Biotech. Phospho-IκBα specific antibody was obtained from New England Biolabs. Anti-FLAG antibodies were purchased through Sigma. MG132, lactacystin and staurosporin were from Calbiochem.

Plasmids and Proteins. Ub and various Ub mutants were expressed in a modified E. coli strain, BL21(DE3)pJY2, to prevent misincorporation of lysines (You et al., 1999). E1 was purified from calf thymus by covalent affinity chromatography on Ub-Sepharose. Expression plasmids for GST-Ubch5c and GST-Ubc13 were kindly provided by Dr. Alan Weissman (NCI). IκBα and mutants were in vitro translated in wheat germ extract in the presence of ³⁵S-methionine (Chen et al., 1995). IKK complex was purified form HeLa cells or from calf thymus using ATP-Sepharose (Upstate Biotechnology Inc.; Spencer et al., 1999). MEKK1 protein was expressed in baculovirus-infected Sf9 insect cells and purified as described previously (Lee et al, 1997). cDNAs for TRAF6 and a truncated form of TRAF6 fused to gyrase B (T6RZC) were gifts from Dr. Jun Ichiro-Inoue (University of Tokyo). These cDNA were subcloned into pFast-Bac (Gibco-BRL) for expression in Sf9 cells as His₆-tagged proteins. While both TRAF6 and T6RZC work indistinguishably in IKK activation in vitro, T6RZC consistently gave better expression and was used in place of TRAF6 in most experiments. Site-directed mutagenesis of the RING finger of TRAF6 at Cys-70 (C70A) and Cys-85/His-87 (C85A/H87A) was carried out using the QuikChange kit (Stratagene). NEMO cDNA was cloned from human placenta cDNA library by Polymerase Chain Reaction (PCR), and then subcloned into pFast-Bac for expression in Sf9 cells. All constructs were verified by automatic DNA sequencing. His₆-tagged proteins were purified using nickel columns (Qiagen).

Cell Culture, Transfection, and Reporter Gene Assays. 70Z and 1.3E2 (NEMO-deficient) cells were kindly provided by Dr. Carol Sibley (University of Washington). These cells were cultured in RPMI1640 supplemented with 10% fetal calf serum (FCS). Human embryonic kidney 293 cells were cultured in DMEM plus 10% FCSTranfection of 293 cells were carried out according to the calcium phosphate precipitation method as previously described (Spencer et al., 1999). Dual luciferase assays were carried out using a kit obtained from Promega. A Renilla luciferase gene driven by the constitutive CMV promoter was included in all transfection experiments as an internal control to correct for transfection efficiency.

In vitro Assay for IKK Activation. To detect endogenous IKK activation, cell extracts (5 mg/ml) were incubated with TRAF6 (or, T6RZC, 0.1 μM) together with an ATP regenerating buffer (Chen et al., 1995). After incubation at 30° C. for 1 hour, the reaction was analyzed by immunoblotting with a phospho-IκBα specific antibody. To identify intermediary factors involved in TRAF6-mediated IKK activation, the inventors used a reconstitution assay by adding ³⁵S-IκBα (0.5 μl) and purified IKK complex (5 nM) to a 5 μl reaction mixture that also contains ATP (2 mM), TRAF6 (0.1 μM), and column fractions (0.5-1 μl). In most IKK activation assays, E1 (0.1 μM) and Ub (0.1 mM) were also added to enhance the reaction, although they can be supplied from the wheat germ extracts used for in vitro translation of IκBα. As an assay for purification of TRIKA1, reaction mixtures also contain 0.5 μg TRIKA2/FrIIa, a fraction eluted from Q-Sepharose with 0.2M NaCl. The reaction was carried out at 30° C. for 1 hour, and then analyzed using a PhosphorImager (Molecular Dynamics) following SDS-PAGE.

Purification of TRIKA1 from HeLa Cytosolic Extracts and Peptide Mass Fingerprinting. All procedures were carried out at 4° C. HeLa cells from 50 liters of suspension culture were resuspended in 70 ml of hypotonic buffer (10 mM Tris-HCl, pH 7.5, 1.5 mM MgCl₂, 10 mM KCl, 0.5 mM DTT, 0.5 mM PMSF), and then lysed in a Dounce homogenizer. Cell debris was removed by ultracentrifugation at 100,000×g for 1 hour. The cleared supernatant (S100) was applied to a Q-sepharose column (60-ml bed volume) pre-equilibrated with Buffer B (20 mM Tris-HCl, pH 7.5, 0.5 MM DTT, 0.5 mM PMSF, 1 μg/ml leupeptin, 0.1M NaCl). The flow-through (containing TRIKA1) was subjected to ammonium sulfate precipitation (40-80%), followed by dialysis against Buffer C (50 mM Hepes-OH, pH 6.5, 0.5 mM PMSF, 0.5 mM DTT) overnight. The dialyzed proteins were loaded onto an SP cation exchange column (5-ml bed volume) using a Fast Performance Liquid Chromatography (FPLC, Pharmacia) system. Bound proteins were eluted with a 100-ml linear gradient of NaCl (0-250 mM). Fractions that contain TRIKA1 activity (90 mM NaCl) were pooled and concentrated before loading onto a Superdex-75 gel filtration column (24-ml bed volume, Pharmacia), which was pre-equilibrated with Buffer D (50 mM Tris-HCl, pH 7.5, 0.5 mM DTT, 0.5 mM PMSF, 150 mM NaCl). After elution with Buffer D, fractions containing the TRIKA1 activity were pooled and loaded directly onto a hydroxylapatite column (2 ml bed volume, Bio-Rad) pre-equilibrated with Buffer E (20 mM Hepes, pH 6.8, 0.5 mM DTT). The column was eluted with a 20-ml gradient of potassium phosphate buffer (0-200 mM) at pH 6.8. Fractions that contain TRIKA1 activity (80 mM phosphate) were adjusted to pH 8.5 with 1M Tris base, and then applied to a MonoQ column (1 ml, Pharmacia) pre-equilibrated with Buffer F (20 mM Tris-HCl, pH 8.5, 0.5 mM DTT, 0.5 mM PMSF, 0.02% Chaps) plus 50 mM NaCl. The column was eluted with a 20 ml gradient of NaCl (50-300 mM) in Buffer F. The fractions corresponding to the peak of TRIKA1 activity contain predominantly two proteins with molecular weights of approximately 16 kDa and 19 kDa as judged by silver staining. These bands were excised for in-gel digest with trypsin. Peptide masses were acquired by matrix-assisted laser desorption/ionization (MALDI) mass spectrometry using a Voyager DE time-of-flight (TOF) spectrometer (PE Biosystems).

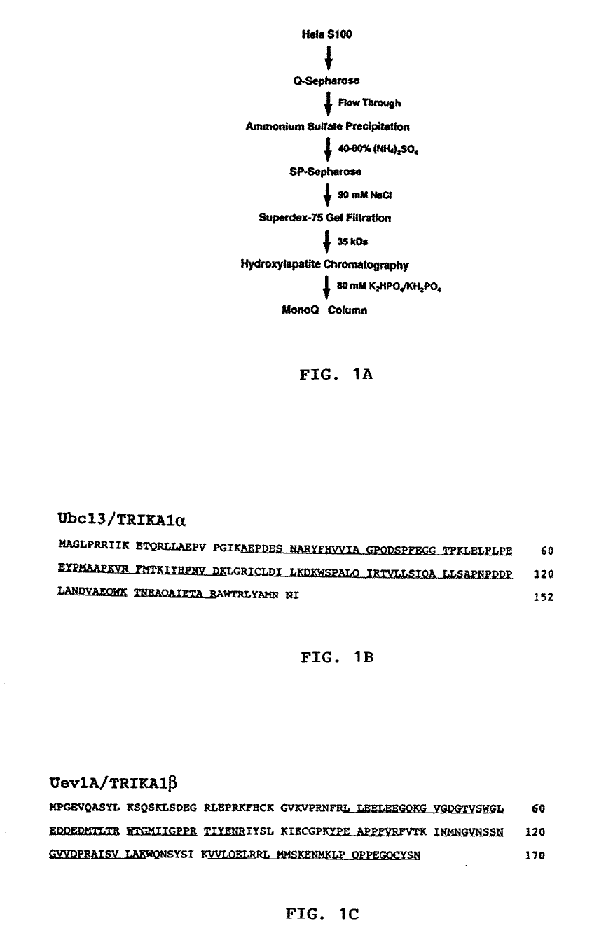

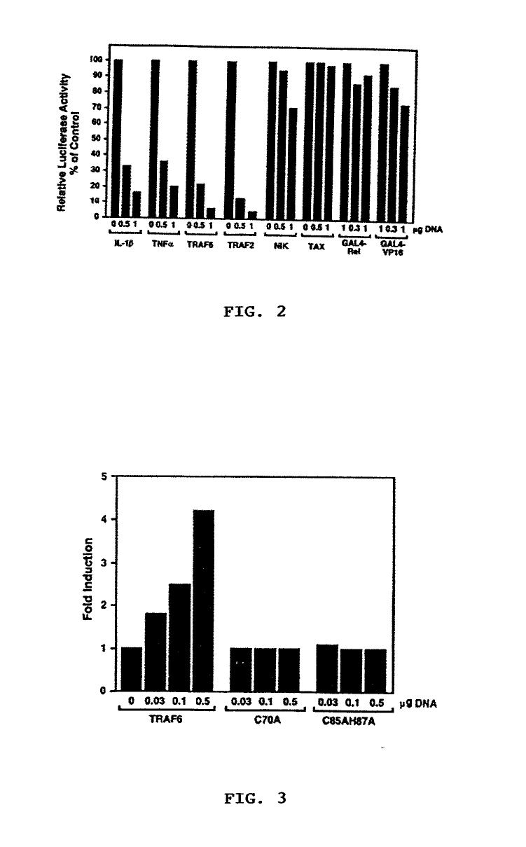

Cloning and Expression of Ubc13 and Uev1A, and Generation of Antisera. Ubc13 and UEV1A were cloned from human placenta cDNA library (Clontech) by PCR with primers designed according to published sequences (Ubc13: D83004; Uev1A/CROC-1A: U39360). The PCR fragments were cloned into the NdeI and BamH1 sites of pET14b (Novagen) in frame with the N-terminal His₆ tag for expression in E.coli. While the sequence of Ubc13 was in complete agreement with the published sequence, the sequence of Uev1A differed from the published sequence (U39360) in two nucleotides (deletion of a G at position 93 and insertion of a C at position 116), which results in a replacement of 6 amino acids (SKSPSQ to VKVPRN; residues 32-37 in FIG. 1C). This difference was unlikely due to PCR or cloning mutation, because the inventors found more than 30 independent EST sequences that matched exactly with the inventors' sequence. Human Mms2 also contains this VKVPRN sequence at the N-terminus, and the inventors found that Mms2 functioned indistinguishably from Uev1A in IKK activation (data not shown).

Site-directed mutagenesis of Ubc13 at Cys-87 (C87A) was carried out using the QuickChange kit (Stratagene). Both the wild type and mutant Ubc13 were subcloned into pEF-IRES-P for expression in mammalian cells under the control of the strong EF1α promoter. For expression of recombinant Ubc13 and Uev1A, pET14b-Ubc13 and pET14b-Uev1A were transformed into BL21(DE3)pLys (Novagen), respectively. Following induction with IPTG, cell extracts were loaded onto a Ni-NTA column (1 ml, Qiagen) to purify the His₆-tagged proteins. For antibody production, 2 mg of purified His₆-Ubc13 and His₆-Uev1A were used to immunize rabbits in the facility of Rockland Inc (Gilbertsville, Pa.).

Depletion of endogenous Uev1A with GST-Ubc13-Sepharose. 100 μl of 293 cell extracts (9 mg/ml) was mixed with 20 μl of glutathione-Sepharose beads preloaded with 80 μg of GST-Ubc13 or the same amount of GST (control). After mixing end-to-end at 4° C. for 1 hour, the beads were removed by centrifugation, and the supernatant was mixed with another 20 μl of GST-Ubc13 or GST beads as described above. The supernatant from the second precipitation was collected for IKK activation assay.

Ubiquitination Assay. For the synthesis of polyUb chains, the reaction mixture contained E1 (0.1 μM), Ubc13/Uev1A (0.4 μM), TRAF6 (0.1 μM), Ub (0.1 mM), and an ATP regenerating system (see above). The reaction was carried out at 30° C. for 1 hour and then resolved by SDS-PAGE. Ubiquitinated products were detected by immunoblotting with an Ub-specific antibody (P4D1).

### EXAMPLE 2

### Results

A cell-free system that activates IKK in response to TRAF6. Addition of recombinant TRAF6 to cell extracts prepared from 70Z pre-B cells led to the phosphorylation of endogenous IκBα that could be detected with a phospho-IκBα specific antibody. This reaction is strictly dependent on NEMO, since cell extracts prepared from 1.3E2, a NEMO-deficient line derived from 70Z cells (Courtois et al, 1997), failed to phosphorylate IκBα in the presence of TRAF6. Moreover, addition of recombinant NEMO to the 1.3E2 extract restored IκBα phosphorylation in response to TRAF6.

NF-κB agonists trigger the phosphorylation of IκBα at both serines 32 and 36. To determine whether IκBα is phosphorylated at these serine residues in the in vitro TRAF6-inducible system, the inventors tested in vitro translated, ³⁵S-labeled IκBα serine mutants in the inventors' assay. While wild-type IκBα was fully phosphorylated, as indicated by the electrophoretic mobility shift, mutation at both serines 32 and 36 abolished this phosphorylation. Single point mutation at either S32 or S36 led to an intermediary gel shift, which is indicative of single site phosphorylation at the remaining wild-type serine residue. Therefore, the in vitro TRAF6-inducible system retains the specificity of IκBα phosphorylation.

TRAF6-dependent activation of IKK requires intermediary factors. To determine whether TRAF6 activates IKK directly, the inventors purified the IKK complex from unstimulated HeLa cells or from calf thymus through several chromatographic steps, including an ATP-affinity column step. The purified IKK complex from HeLa cells contains IKKα, IKKβ, and NEMO, and can be activated directly by MEKK1 (Lee et al., 1998). The ability to purify this latent IKK complex from HeLa cells allows the inventors to search for upstream factors required for its activation. One of the candidate upstream activators is TRAF6. However, unlike MEKK1, TRAF6 did not activate the purified IKK complex directly. In contrast, the crude extract from 293 cells can be activated by TRAF6, suggesting the existence of intermediary factors required for IKK activation by TRAF6 in the extract. As an initial step to identify these factors, the inventors fractionated HeLa cytosolic extract (S100) on Q-Sepharose through step elution with increasing concentration of NaCl. Significantly, the activation of the IKK complex by TRAF6 requires at least two fractions, the unbound fraction (Fr.I) and the 0.1-0.2M NaCl eluate (Fr.IIa). Omission of either fraction abolishes IKK activation by TRAF6. The factor present in Fr.I is hereby referred to as TRAF6-Regulated IKK Activator 1 (TRIKA1), whereas the factor present in Fr.IIa is referred to as TRIKA2.

Purification and identification of TRAF6-Regulated IKK Activator 1 (TRIKA1). The inventors have purified TRIKA1 from HeLa cytosolic extracts to apparent homogeneity through six steps of conventional chromatography (FIG. 1A). Silver staining of fractions from the last MonoQ step, and the IKK stimulatory activity assay of the same fractions was performed. This factor has an apparent molecular size of 35 kDa based on gel filtration chromatography, and is separated into two bands on SDS-PAGE with molecular sizes of 16- and 19-kDa, respectively. The intensity of these bands correlated with IKK-stimulatory activity, indicating that they are candidate subunits of TRIKA1 (α and β subunits).

The 16- and 19-kDa bands were excised for mass spectrometric analysis following digestion with trypsin. Peptide mass fingerprinting (MALDI-TOF) of the 16-kDa protein identified 9 peptides whose masses matched those predicted from human Ubc13, an Ub conjugating enzyme (FIG. 1B). The 19-kDa protein has 11 peptides whose masses matched those of an isoform of human Uev1 such as Uev1A (FIG. 1C), a Ub conjugating E2 enzyme variant (UEV) that is structurally related to E2 enzymes but lacks the catalytic cysteine residue found in a typical E2 (Sancho et al., 1997; Rothofsky and Lin, 1997).

Recombinant Ubc13/Uev1A complex supports the activation of IKK by TRAF6. To verify that Ubc13 and Uev1A are indeed components of TRIKA1, the inventors expressed these proteins in E. coli and purified the recombinant proteins to apparent homogeneity with the aid of a hexa-histidine (His₆) affinity tag. Recombinant Ubc13 and Uev1A form a heterodimer that migrates at approximately 45 kDa on a gel filtration column. In the presence of TRAF6, the recombinant Ubc13 and Uev1A activated IKK in a manner that correlated with the formation of the heterodimer. Furthermore, mutation of the active site Cys-87 of Ubc13 (C87A) abolished its ability to support IKK activation by TRAF6. Like the native TRIKA1, recombinant Ubc13/Uev1A stimulated IκBα phosphorylation by IKK at both serines 32 and 36. The inventors also tested whether other E2s could support TRAF6-mediated IKK activation. Among several E2s tested, only Ubc13/Uev1A was capable of activating IKK together with TRAF6. These results confirm that Ubc13/Uev1A is the TRIKA1 that links TRAF6 to IKK activation.

Ubc13/Uev1A is required for IKK activation by TRAF6. The tight binding between Ubc13 and Uev1A makes it feasible to deplete endogenous Uev1A from crude cell extracts with immobilized GST-Ubc13, thus providing an opportunity to address the consequence of removing Uev1A. This is particularly useful in light of the fact that the antibodies generated against Ubc13 or Uev1A fail to immunoprecipitate the respective proteins, although they can detect the proteins by immunoblotting. Cell extracts were devoid of Uev1A after passage through GST-Ubc13-Sepharose, whereas GST-Sepharose did not retain any Uev1A from the extracts. Consequently, cell extracts depleted of Uev1A are severely defective in IKK activation in the presence of TRAF6, whereas control extracts retain full IKK activation potential. Addition of recombinant Ubc13/Uev1A to the Uev1A-depleted extracts restored IKK activation in response to TRAF6. These results indicate that Ubc13/Uev1A is required for IKK activation by TRAF6 in vitro.

To determine whether Ubc13/Uev1A is required for NF-κB activation in living cells, the inventors transfected an expression construct encoding the Ubc13(C87A) mutant into 293 cells together with a reporter gene that expresses luciferase under the control of three tandem repeats of NF-κB binding sites (FIG. 2). The transfected cells were stimulated with IL-1β, TNFα, or by cotransfection with expression constructs encoding TRAF6, TRAF2, NIK, or the TAX protein of the human T cell leukemia virus (HTLV1). As a control, the expression of a luciferase reporter gene driven by Gal4 was also examined, using hybrid transcription activators composed of the DNA binding domain of Gal4 and the transactivation domain of c-Re1 (Gal4-Re1) or VP16 (Gal4-VP16). Remarkably, overexpression of Ubc13(C87A) led to potent inhibition of NF-κB activation by IL-1β, TNFα, TRAF6 or TRAF2. In contrast, Ubc13(C87A) had little effect on NF-κB activation by NIK or TAX, or on Gal4-dependent transcription by Re1 or VP16. Thus, Ubc13/Uev1A appears to be involved primarily in the pathways that require TRAF proteins (TRAF2 for TNFα, and TRAF6 for IL-1β). Taken together, these results strongly suggest that Ubc13/Uev1A is required for NF-κB activation by TRAF proteins in the IL-1 and TNF pathways.

TRAF6 facilitates the assembly of K63-linked polyUb chains in conjunction with Ubc13/Uev1A. Previous studies have shown that Ubc13 and Mms2, a yeast homolog of Uev1A, form an E2 complex to catalyze the synthesis of unique polyUb chains linked through lysine-63 (K63) of ubiquitin (Hofmann and Pickart, 1999). This finding, together with the inventors' present results that this E2 complex is involved in IKK activation by TRAF6, raises the possibility that TRAF6 may also be involved in ubiquitination. In fact, TRAF6 contains a RING finger domain and five repeats of zinc fingers at its N-terminus (Cao et al., 1996). Several other RING finger proteins have previously been shown to function as ubiquitin ligases in polyubiquitination (Lorick et al., 1999). To test whether TRAF6 has a role in ubiquitination, the inventors carried out an in vitro ubiquitination assay in the presence of E1, Ubc13/Uev1A (as an E2), ATP, and Ub. When all components were present, polyUb chains synthesis was readily detectable with an Ub-specific antibody. In the absence of any of the components, no significant polyubiquitination was observed. (The Ub₂ is principally a contaminant in the commercial Ub). Inclusion of an Ub mutant (KO) in which all seven lysines were mutated to arginine prevented polyUb chain formation. Restoration of a lysine at position 48 (K48) on an otherwise lysine-less background was not sufficient to restore polyubiquitination. In contrast, restoration of a lysine at position 63 (K63) restored polyubiquitination. These results show that TRAF6 facilitates the synthesis of polyUb chains through K63 rather than K48. In this regard, TRAF6 is an Ub ligase (E3) that partners with Ubc13/Uev1A (E2). The inventors also found that TRAF2 functioned together with Ubc13/Uev1A to catalyze the synthesis of K63-linked polyUb chains, consistent with the obligatory role of Ubc13/Uev1A in NF-κB activation by TNFα and TRAF2 (FIG. 2).

To determine whether the RING finger domain of TRAF6 is important for its Ub conjugating activity, the inventors mutated the highly conserved cysteine and histidine residues in the RING domain to generate two point mutants, C70A and C85A/H87A. These constructs were expressed in 293 cells as Flag-tagged proteins and immunopurified for ubiquitination assays. While wild-type TRAF6 catalyzed the assembly of K63-linked polyUb chains from a K63 Ub mutant in the presence of E1 and Ubc13/Uev1A, the RING finger mutations abolished this Ub conjugating activity. These same mutations also abolished the ability of TRAF6 to activate the expression of an NF-κB reporter gene (luciferase) when overexpressed (FIG. 3), suggesting that the NF-κB-inducing activity of TRAF6 is linked to its Ub conjugating activity through the use of the RING domain.

K63-linked polyUb chains are involved in IKK activation by TRAF6. The inventors' findings that TRAF6 and Ubc13/Uev1A are involved in IKK activation, and that they catalyze the synthesis of K63-linked polyUb chains, led the inventors to investigate the role of these unique Ub chains in IKK activation. The inventors first examined a panel of Ub mutants for their ability to activate IKK in vitro (FIG. 4). These mutants include a lysine-less mutant (KO), single-lysine mutants, which contain one lysine with the remaining six lysine residues mutated to arginine (K6, K11, K27, K29, K33, K48 and K63), and single K/R mutants, which contain a single point mutation from lysine to arginine with the remaining lysines intact (R11, R29, R48 and R63). In addition, the inventors also tested methylated Ub (MeUb), in which all seven lysine residues were blocked by methylation, thus preventing elongation of Ub chains. Strikingly, MeUb, KO, and all single-lysine mutants except for K63 fail to support IKK activation by TRAF6 indicating that K63 is the only important lysine on Ub required for this function. In further support of this conclusion, a single point mutation at position 63 from Lys to Arg (R63) abolished the stimulatory activity of Ub, whereas mutations at other lysines had no effect. These results strongly suggest that K63-linked polyUb chains play an important role in TRAF6-mediated IKK activation.

To determine whether the assembly of K63-linked polyUb chains is required for IKK activation by TRAF6 in crude cell extracts, the inventors tested several Ub lysine mutants for their ability to interfere with IKK activation in the extracts. Addition of wild-type or K63 Ub had no effect on endogenous IκBα phosphorylation stimulated by TRAF6. In contrast, Ub mutants lacking any lysine (KO) or retaining only one lysine at position 48 (K48) exert a dominant negative effect on endogenous IKK activation by TRAF6. These mutants did not interfere with IKK activation triggered by MEKK1 or IKKβ. Taken together, these results further strengthen the conclusion that TRAF6-mediated activation of IKK requires the assembly of K63-linked polyUb chains.

The critical role of K63 rather than K48 of Ub in IKK activation raises the question of whether proteasomal degradation plays any role at all in IKK activation, since K48-linked polyUb chains are preferentially recognized by the proteasome (Chau et al., 1989). In the inventors' in vitro reconstitution system, no proteasome was added to the assay, suggesting that IKK activation does not require proteasome. To rule out the possibility that any residual proteasomal activity might be required for IKK activation, the inventors added proteasome inhibitors MG132 or lactacystin to the assay. No inhibition of IKK activation was observed in the presence MG132 or lactacystin at 20 μM, a concentration known to completely block proteasomal activity (Chen et al., 1995). As a control, the kinase inhibitor staurosporin (STS) significantly inhibited IKK activity in the extract. This experiment, together with previous results that proteasome inhibitors do not interfere with IκB phosphorylation in vivo (Palombella et al., 1994), shows that IKK is activated by TRAF6 through a mechanism involving K63-linked polyUb chains, but not proteasomal degradation.

This invention reports, the purification and identification of a protein complex that links IKK to its upstream activator TRAF6. This complex is composed of Ubc13 and Uev1A (Mms2), a dimeric ubiquitin-conjugating enzyme previously shown to catalyze the synthesis of K63-linked polyubiquitin chains (Hofmann and Pickart, 1999).

The involvement of Ubc13/Uev1A as an ubiquitin-conjugating enzyme in TRAF6-mediated IKK activation led to the examination of the potential role of TRAF6 in ubiquitination. TRAF6 greatly facilitated the synthesis of K63-linked polyUb chains by Ubc13/Uev1A. This E3 activity of TRAF6 requires an intact RING domain, a structural motif that is also critical for TRAF6 to induce NF-κB in cells.

The invention further reports that K63-linked polyUb chains play an important regulatory role in IKK activation, based on the following lines of evidence. First, a single point mutation at K63, but not at other lysine residues, abolishes the ability of Ub to support IKK activation by TRAF6; second, restoration of a single lysine at position 63, but not at any other position, is sufficient to activate IKK in a reconstituted system; third, Ub mutants that fail to assemble into K63-linked polyUb chains exert dominant negative effects on IKK activation in cell extracts; fourth, inhibition of the proteasome has no effect on IKK activation. These results, together with the aforementioned findings that IKK activation requires the catalytic cysteine of Ubc13 and the RING domain of TRAF6, provide strong support for a critical role of K63-linked polyUb chains in IKK activation.

### EXAMPLE 3

### Materials & Methods

Cells and Reagents. HeLa S3 cells were purchased from National Culture Center (Minneapolis, Minn.). The TAB2 antibody was produced from rabbits by immunization using a His₆-TAB2 N-terminal fragment (residues 1-450) as an antigen, and then affinity purified on a TAB2 column. Antibodies against TAK1, TAB1, ubiquitin, IκBα were purchased from Santa Cruz Biotech. The antibody against phospho-JNK was obtained from New England Biolabs. cDNAs encoding TAK1, TAB 1, TAB2, and MKK6 were cloned by PCR from cDNA libraries based on published sequences (Deng, et al., 2000; Takaesu et al., 2000; Raingeaud et al., 1996) and verified by sequencing. pcDNA3-JNK, -cjun and -cjun (S63A/S73A) were provided by Dr. Frank Lee (Lee et al., 1997). cDNAs encoding TRAF6, Ubc13, Uev1A, and IκBα have been previously described (Deng et al., 2000). In vitro translation of IκBα and cJun were carried out using wheat germ extracts supplemented with ³⁵S-methionine (Chen et al., 1996). ³⁵S-IκBα, which has a FLAG tag at its N-terminus, was further purified by immunoaffinity chromatography. Site-directed mutagenesis was carried out using the QuikChange kit (Strategene). Recombinant MKK6 and JNK were expressed in E. coli as His₆-tagged proteins and purified using nickel columns. TAK1, TAB1, and TAB2 were expressed in insect (Sf9) cells individually or in combination following infection or co-infection of baculovirus harboring the respective genes in pFAST-Bac (Gibco-BRL), and then purified via nickel affinity column followed by MonoQ or gel filtration chromatography. Similarly, recombinant IKKβ(K44M), IKKβ(K44M/S177A/S181) and Nemo were expressed in Sf9 cells, except that these proteins were engineered to contain a FLAG epitope at their N-termini and were purified to homogeneity by FLAG immunoaffinity chromatography. Other reagents, including E1, TRIKA1 (E2), TRAF6 (E3) and various Ub mutants have been described previously (Deng et al, 2000; Hofmann et al., 1999).

Purification of TRIKA2. Crude cytosolic extracts (S100) from 100-liter culture of HeLa cells were prepared as previously described (Deng et al, 2000), and applied to a Q-Sepharose column pre-equilibrated with buffer A (20 mM Tris-HCl, pH 7.5, 1 mM DTT, 0.5 mM PMSF, 1 μg/ml leupeptin) containing 0.1 M NaCl. Proteins bound to the column were eluted with Buffer A plus 0.21 M NaCl, and then precipitated with ammonium sulfate (40%). The pellets were resuspended and loaded onto a Phenyl-Sepharose column. Bound proteins were eluted with a gradient of decreasing concentration of ammonium sulfate (0.5 M-0 M) in Buffer A. The fractions containing TRIKA2 activity were applied to a Heparin-Sepharose column and eluted with a gradient of NaCl (0-0.3M) in Buffer A. Active TRIKA2 fractions were concentrated before loading onto a Superdex-200 column preequilibrated with buffer B (20 mM Tris-HCl, pH 7.5, 0.5 mM DTT, 0.2 mM PMSF, 0.15 M NaCl, 15% Glycerol). Proteins eluted from the Superdex column were applied to a Mono-S column and eluted with a gradient of NaCl (0-0.3M) in Buffer C (20 mM Hepes, pH 7.0, 0.5 mM DTT, 0.2 mM PMSF, 0.02% Chaps, 10% Glycerol). Fractions containing TRIKA2 were diluted in buffer D (20 mM Tris-HCl, pH 7.5, 0.5 mM DTT, 0.2 mM PMSF, 0.02% Chaps, 10%Glycerol), loaded onto a Mono-Q column, and then eluted with a gradient of NaCl (0.1-0.3M) in Buffer D. For immunopurification of TRIKA2 cell extracts were prepared as described for S100 except that HeLa cells were lysed in Buffer A supplemented with 0.5% NP40 and 0.1M NaCl. TRIKA2 was partially purified by conventional chromatographic steps including Q-Sepharose, ammonium sulfate precipitation, Heparin-Sepharose and MonoQ as described above. The fractions containing TRIKA2 activity from MonoQ were further purified by immunoprecipitation using the TAB2 antibody immobilized on Protein A-Sepharose.

Kinase Assays. IKK activity was measured in a reconstituted system (10 μl) containing an ATP buffer (50 mM Tris-HCl, pH 7.5, 5 mM MgCl₂, 2 mM ATP), ³⁵S -IκBA (11), purified IKK complex (5 nM), E1 (50 nM), Ubc13/Uev1A (0.3 μM), TRAF6 (0.1 μM), Ub (50 μM), and various amounts of partially purified or immunopurified TRIKA2/TAK1 complex. Following incubation at 30° C. for 1 hour, the reaction products were resolved by SDS-PAGE and analyzed using a Phosphorlmager.

To assay the activity of TAK1, the kinase complex immobilized on the anti-TAB2 beads was incubated with E1, Ubc13/Uev1A, TRAF6, Ub and the ATP buffer at room temperature for 1 hour under conditions similar to those used for IKK activation (see above). The beads were then washed extensively with TBS (20 mM Tris-HCl, pH 7.5, 150 mM NaCl) plus 0.5% NP40, followed by incubation with recombinant MKK6 (1 μM), γ-³²P-ATP (0.5 μCi/μl), and a kinase buffer (50 mM Tris-HCl, pH 7.5, 0.5 mM DTT, 5 mM MgCl₂, 50 μM ATP). Phosphorylation of IKKβ was carried out similarly, except that IKKβ was preincubated with Nemo to form a complex which then serves as a substrate for TAK1. The reactions were carried out at 30° C. for 1 hour and then analyzed by SDS-PAGE. For assays of JNK activity, the Ub-activated TAK1 beads were incubated with MKK6 (60 nM), JNK (0.1 μM), ³⁵S-cJun or cJun (S63A/S73A, 1 μl), and the ATP buffer. Phosphorylation of cJun was then determined based on its mobility shift on SDS-PAGE.

Generation of TRAF6-gyraseB stable cell lines. The TRAF6-gyrase B fusion was constructed by replacing the C-terminal TRAF domain of TRAF6 (residues 1-358) with the N-terminal fragment of bacterial gyrase B (T6RZC). Another chimeric construct contained residues 1-291 of TRAF6 (T6RZ). These chimeras were cloned into the mammalian expression vector pEF-IRES-P (Deng et al., 2000). Following transfection of DNA into 293 cells, stable clones were selected in puromycin (1 μg/ml). To induce the dimerization of TRAF6 and subsequent activation of the JNK and IKK pathways, cells were treated with coumermycin A (1 μM) for different lengths of time, and then cell extracts prepared for further analysis.

### EXAMPLE 4

### Results

Identification of TRIKA2 as TAK1-TAB1-TAB2 complex. To facilitate the purification of TRIKA2 which might contain TAB2, the inventors prepared HeLa cell extracts in a buffer containing 0.5% NP40. From these extracts, the inventors used a TAB2-specific antibody to purify a complex containing stoichiometric amounts of TAK1, TAB1 and TAB2 as visualized by colloidal blue staining and immunoblotting. This immunopurified complex contained a bona fide TRIKA2 activity in that it stimulated IKK only in the presence of both Ubc13/Uev1A and TRAF6 (together with other ubiquitination components including E1 and Ub).

To determine whether the TAK1/TAB1/TAB2 complex is indeed TRIKA2, the inventors transfected expression vectors harboring FLAG-tagged TAK1 and TAB2 cDNA, respectively, into 293 cells, and then immunopurified these proteins using a FLAG-specific antibody. In each case, the transfected protein associated with endogenous partners to form a TAK1/TAB1/TAB2 complex, which activated IKK, but only in the presence of both Ubc13/Uev1A and TRAF6. Importantly, a point mutation in the ATP binding domain of TAK1 (K63W) that abolished its kinase activity also abrogated its ability to stimulate IKK.

Since the catalytic activity of TAK1 was essential for IKK activation, the inventors examined whether TAK1 functioned as an IKK kinase (IKKK) to directly phosphorylate IKK at two specific serines (S177 and S181) in the activation loop. To activate endogenous TAK1, the inventors immunoprecipitated the TAK1 complex from HeLa cell extracts, which was then subjected to TRAF6-mediated ubiquitination reactions in the presence or absence of Ubc13/Uev1A. This TAK1 complex was then incubated with γ-³²p-ATP together with recombinant IKKβ proteins, which include a catalytically inactive mutant (K44M; to prevent IKKβ autophosphorylation) and a triple-point mutant in which two serines in the activation loop are also mutated (K44M/S177A/S181A). Significantly, the TAK1 kinase, once activated by Ubc13/Uev1A-mediated ubiquitination, was able to phosphorylate IKK specifically at serines 177 and 181. Thus, TAK1 is a ubiquitin-dependent kinase of IKKβ.

TAK1 complex is both sufficient and necessary to activate IKK in conjuction with TRAF6 and Ubc13-Uev1A. To address the role of the individual components of the TAK1/TAB1/TAB2 complex in IKK activation, the inventors expressed TAK1, TAK1/TAB1, TAK1/TAB2 or co-expressed all three proteins in insect cells (Sf9) using the baculovirus expression system. These proteins were purified and then tested for IKK activation under the condition where the amount of TAK1 was equivalent. Only TAK1/TAB2 and TAK1/TAB1/TAB2 were able to stimulate IKK in the presence of Ubc13/Uev1A and TRAF6, suggesting that TAK1/TAB2 is the minimal sub-complex capable of activating IKK.

The above experiments were carried out using an IKK complex partially purified from HeLa cell extracts (>60% pure). To determine whether the TAK1 complex could activate IKK in a reconstituted system using highly purified proteins, the inventors further purified the IKK complex to homogeneity by immunoprecipitation using a Nemo-specific antibody. The purified IKK complex contained exclusively IKKα, IKKβ and Nemo/IKKγ based on silver staining and immunoblotting. The inventors also obtained highly purified recombinant TAK1 complex (from Sf9 cells) and 35S-IκBα. Using these purified proteins, together with purified E1, E2 (Ubc13/Uev1A), and E3 (TRAF6), the inventors were able to reconstitute IKK activation by TRAF6 in a manner that was dependent on Ubc13/Uev1A (TRIKA1) and TAK1/TAB1/TAB2 (TRIKA2).

To determine whether the TAK1 complex is essential for IKK activation by TRAF6, the inventors immunodepleted this complex from 293 cell extracts using a TAB2 antibody. Removal of TAB2 from cell extracts abolished IKK activation by TRAF6, whereas immunoprecipitation with a control IgG antibody had no effect. Furthermore, addition of recombinant TAK1/TAB2 back into the depleted cell extracts restored IKK activation.

TAK1 is a ubiquitin-dependent kinase of the MKK-JNK pathway. Previous studies have shown that TRAF6 is essential for both IKK and JNK activation (Cao et al., 1996; Ishida et al., 1996; Lomaga et al., 1999; Naito et al., 1999), and that TAK1 can phosphorylate MKK6 (Ninomiya et al., 1999) which in turn activates the JNK/p38 kinase pathway (Raingeaud et al., 1996; Davis, 2000) The inventors' finding that IKK is activated by TAK1 in a TRAF6- and Ubc13/Uev1A-dependent manner raises the interesting possibility that the activation of MKK6 and subsequent activation of the JNK pathway might also be dependent on ubiquitin-activation of TAK1. Indeed, when the endogenous TAK1 complex was subjected to ubiquitination by TRAF6 and Ubc13/Uev1A, it was activated to phosphorylate MKK6. The Ub-activated TAK1 kinase specifically phosphorylates MKK6 at Ser-207 and Thr-211 in the activation loop (Raingeaud et al., 1996; Davis, 2000) allowing MKK6 to stimulate the kinase activity of JNK, which phosphorylates c-Jun at serines 63 and 73. In further support of the role of Ub in MKK/JNK activation, addition of methylated ubiquitin (MeUb), which blocks polyubiquitination (Hershko et al., 1985) to cell extracts abolished the activation of both JNK and IKK by TRAF6. Thus, Ub-activation of TAK1 provides a unifying mechanism for coordinate activation of the IKK and JNK pathways.

TRAF6 and Ubc13/Uev1A catalyze the synthesis of a unique polyubiquitin chain linked through lysine-63 (K63) of Ub, and the formation of such a chain is essential for IKK activation via a mechanism that does not involve proteasomal degradation (Deng et al., 2000) To determine whether the formation of K63 chains is also important for TAK1 activation, the inventors subjected the immunopurified TAK1 complex to ubiquitination reactions in the presence of various Ub mutants (FIG. 5). Strikingly, a point mutation at position 63 from lysine to arginine (R63) completely abolished the ability of Ub to stimulate TAK1 activity (FIG. 5). Conversely, restoration of a single lysine at position 63 (K63) on an otherwise zero lysine background rescued the ability of Ub to activate TAK1. Thus, a lysine at position 63 is both necessary and sufficient for Ub to activate TAK1, most likely through the synthesis of K63-linked polyUb chains.

K63-linked polyubiquitination of TRAF6. Next, the inventors sought to identify potential targets of ubiquitination, which could include components of the TAK1 complex and TRAF6 itself. To test whether these proteins are potential ubiquitination targets, the inventors treated HeLa cells with IL-1β and then harvested the cell extracts for immunoblotting with antibodies specific for each of these proteins. Interestingly, stimulation of cells with IL-1β led to the accumulation of high molecular weight conjugates of TRAF6 with kinetics that correlated with the degradation and resynthesis of IκBα. That these conjugates were indeed polyubiquitinated TRAF6 was confirmed by immunoprecipitating TRAF6 followed by immunoblotting with a Ub-specific antibody. No detectable ubiquitination of components of the TAK1 complex was observed in the same experiment.

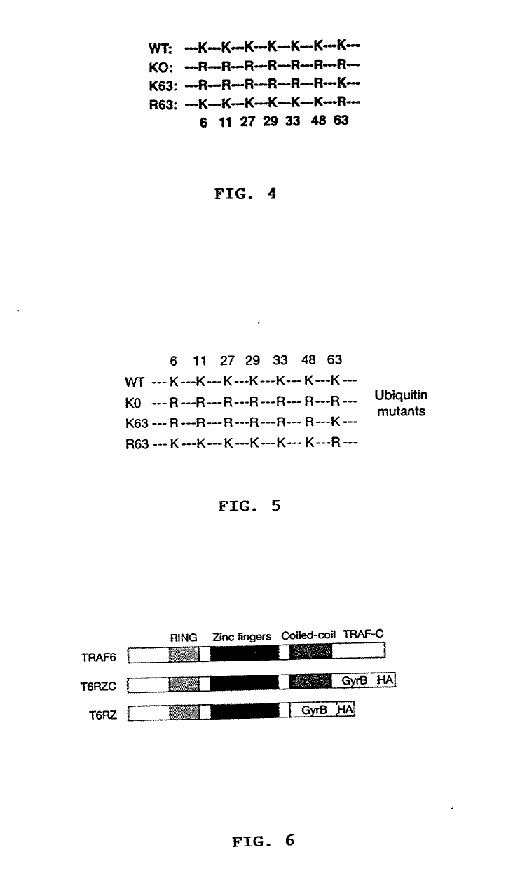

It has been suggested that oligomerization of TRAF6 through its C-terminal TRAF domain leads to the activation of the IKK and JNK pathways (Baud et al., 1999). To determine whether oligomerization of TRAF6 triggers its ubiquitination, the inventors sought to create a cell line in which the activation of IKK is controlled by the inducible oligomerization of TRAF6. The inventors replaced the C-terminal TRAF domain of TRAF6 with a fragment of the bacterial gyrase B, which dimerizes in the presence of coumermycin A (Farrar, 1996) (FIG. 6). The chimeric construct (T6RZC) was transfected into 293 cells to establish a stable line in which IκBα was rapidly degraded upon the addition of coumermycin A. Concomitant with the degradation of IκBα, a ladder of polyubiquitinated T6RZC accumulated. Notably, while treatment of cells with coumermycin A or IL-1β induced the rapid degradation of IκBα, no evidence of T6RZC or TRAF6 degradation was observed during the same time course. Interestingly, deletion of a coil-coiled region (residues 292-358) in TRAF6 (T6RZ) abolishes its ubiquitination as well as its ability to stimulate IκBα degradation, suggesting a correlation between the ability of TRAF6 to be ubiquitinated and its ability to stimulate the IKK pathway. Following ubiquitination, TRAF6 led to the activation of IKK in the presence of the TAK1 complex without further requirements for additional ubiquitination enzymes such as E1 and Ubc13/Uev1A. The polyUb chains on T6RZC were apparently K63-linked, since it was polyubiquitinated by Ub mutants containing K63 but not R63.

The invention also reports the purification and identification of TRIKA2, which turns out to be composed of TAK1, TAB1 and TAB2, a protein kinase complex previously implicated in IKK activation through an unknown mechanism (Ninomiya et al., 1999; Takaesu et al., 2000) Furthermore, the data indicates that the TAK1 kinase complex phosphorylates and activates IKK in a manner that depends on TRAF6 and Ubc13/Uev1A. Moreover, the activity of TAK1 to phosphorylate MKK6, which activates the JNK/p38 kinase pathway, is directly regulated by K63-linked polyubiquitination. Evidence that TRAF6 itself is conjugated by the K63 polyUb chains is provided herein. These results indicate that ubiquitination plays an important regulatory role in stress response pathways, including those of IKK and JNK. The data strongly suggest that TAK1 is an IKKK that phosphorylates and activates IKK in the TRAF6 pathway. Futhermore, the results clearly demonstrate a crucial role of K63-linked polyubiquitination in TAK1 activation.

Ascension numbers for the proteins described in the present invention are provided: XM006284 (TRAF6); XM011774 (TRAF2); XM010000 (TAB1); NM015093 (TAB2); AF241230 (TAK1); AY008273 (UevA1); and XM052540 (Ubc13)

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

IX. References

The following references, to the extent that they provide exemplary procedural or other details supplementary to those set forth herein, are specifically incorporated herein by reference.

Ansieau et al., Proc. Na't. Acad. Sci. USA 93, 14053-14058, 1996.

B. Ardrey, Electrospray Mass Spectrometry, Spectroscopy Europe, 4, 10-18, 1992.

Baeuerle, P. A., and Baltimore, D, Cell 53, 211-217, 1988.

Baud, V., Liu, Z. -G., Bennett, B., Suzuki, N., Xia, Y., and Karin, M. (1999).Genes Dev. 13, 1297-1308.

Brockman, J. A., Scherer, D. C., McKinsey, T. A, Hall, S. M., Qi, X., Lee, W. Y., and Ballard, D. W., (1995). Mol. Cell. Biol. 15, 2809-2818.

Brown, K., Gerstberger, S., Carlson, L., Franzoso, G., and Siebenlist, U. (1995). Science 267, 1485-1488.

Berkenkamp et al., Science, 281(5374):260-2, 1998.

Berzal-Herranz, Joseph, Burke, “In vitro selection of active hairpin ribozymes by sequential RNA-catalyzed cleavage and ligation reactions,” Genes Dev, 6(1):129-134, 1992.

Capaldi, Bell, Branchek, “Changes in order of migration of polypeptides in complex III and cytochrome C oxidase under different conditions of SDS polyacrylamide gel electrophoresis,” Biochem. Biophys. Res. Comm., 74(2):425-433, 1977.

Caspers et al., Mol. Biochem. Parasitol., 47(2):143-150, 1991.

Cech, Zaug, Grabowski, “In vitro splicing of the ribosomal RNA precursor of Tetrahymena: involvement of a guanosine nucleotide in the excision of the intervening sequence,” Cell, 27(3 Pt 2):487-96, 1981.

Cao, Z., Xiong, J., Takeuchi, M., Kurama, T., and Goeddel, D. V., Nature 383, 443-446, 1996.

Cao, Z., Henzel, W. J., Gao, X. Science 271, 1128-31, 1996.

Chau, V., Tobias, J. W., Bachmair, A., Marriott, D., Ecker, D. J., Gonda, D. K., and Varshavsky, A. Science 243, 1576-1583, 1989.

Chen, Z. J., Hagler, J., Palombella, V. J., Melandri, F., Scherer, D., Ballard, D., and Maniatis, T. Genes Dev. 9, 1586-1597, 1995.

Chen, Z. J., Parent, L., and Maniatis, T, Cell 84, 853-862, 1996.

Chen, Z. J., and Maniatis, T. In Ubiquitin and the Biology of the Cell, J. -M. Peters, J. R. Harris, and D. Finley, eds. (New York: Plenum Press), pp.303-32, 1998.

Chen and Okayama, “High-efficiency transformation of mammalian cells by plasmid DNA,” Mol. Cell Biol., 7(8):2745-2752, 1987.

Cheng et al. Science 267, 1494-1498, 1995.

Chowrira, Berzal-Herranz, Burke, “Ionic requirements for RNA binding, cleavage, and ligation by the hairpin ribozyme,” Biochemistry, 32(4):1088-1095, 1993.

Chowrira, Pavco, McSwiggen, “In vitro and in vivo comparison of hammerhead, hairpin, and hepatitis delta virus self-processing ribozyme cassettes,” J Biol Chem, 269(41):25856-25864, 1994.

Courtois, G., Whiteside, S. T., Sibley, C. H., Israel, A. Mol. Cell. Biol. 17, 1441-1449, 1997.

Davis, R. J. Cell 103, 239-52, 2000.

De Jager, Abdel-Nabi, Serafini, Pecking, Klein, Hanna Jr., “Current status of cancer immunodetection with radiolabeled human monoclonal antibodies,” Semin. Nucl. Med., 23(2):165-179, 1993.

Deng et al. Cell 103, 351-361, 2000.

Dinarello, C. A. Blood 87, 2095-2147, 1996.

Doolittle and Ben-Zeev, “Immunodetection of lipoprotein lipase: antibody production, immunoprecipitation, and western blotting techniques,” Methods Mol Biol, 109:215-237, 1999.

Effenhauser, et al. Anal. Chem., 66:2949-2953, 1994.

Effenhauser, et al. Anal Chem., 65:2637-2642, 1993.

European Patent No. 0 273,085

Evan et al., Mol. Cell. Biol. 5:3610-3616, 1985.

Farrar et al., Nature 383, 178-81, 1996.

Fechheimer, Boylan, Parker, Sisken, Patel, Zimmer, “Transfection of mammalian cells with plasmid DNA by scrape loading and sonication loading,” Proc. Nat'l Acad. Sci. USA, 84:8463-8467, 1987.

Fenn et al., J. Phys. Chem. 88, 4451-59, 1984

Fodor et al., Nature, 364:555-556, 1993.

Forster Ann. Phys. 2, 55-75, 1948.

Forster and Symons, “:Self-cleavage of plus and minus RNAs of a virusoid and a structural model for the active sites,” Cell, 49(2):211-220, 1987.

Fraley, Fornari, Kaplan, “Entrapment of a bacterial plasmid in phospholipid vesicles: potential for gene transfer,” Proc. Nat'l Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

Gedrich et al., J. Biol. Chem. 271, 12852-12858, 1996

Gerlach et al., Nature (London), 328:802-805, 1987.

Ghosh, S., May, M. J., and Kopp, E. B. Annu. Rev. Immunol. 16, 225-260, 1998.

Ghosh and Bachhawat, Targeting of Liposomes to Hepatocytes. In: Liver Diseases, Targeted Diagnosis and Therapy Using Specific Receptors and Ligands. Wu et al., eds., Marcel Dekker, New York, pp. 87-104, 1991.

Gopal, Mol. Cell Biol., 5:1188-1190, 1985.

Graham and van der Eb, “A new technique for the assay of infectivity of human adenovirus 5 DNA,” Virology, 52:456-467, 1973.

Grussenmeyer et al., Proc. Nat'l Acad. Sci. USA 82:7952-7054, 1985.

Gulbis and Galand, Hum Pathol, 24(12):1271-85, 1993.

Hacia et al., Nature Genetics, Dec. 14, 1996.

Harland and Weintraub, “Translation of mRNA injected into Xenopus oocytes is specifically inhibited by antisense RNA,” J. Cell Biol., 101: 1094-1099, 1985.

Harrison et al., Science, 261:895-897, 1993.

Haseloff and Gerlach, “Simple RNA enzymes with new and highly specific endoribonuclease activities,” Nature, 334(6183):585-91, 1988.

Hatada et al., Curr Opin Immunol 12(1):52-8, 2000

Haung et al., Proc Nat'l Acad Sci USA. 94(24):12829-32, 1997.

Hershko, A., and Heller, H. Biochem. Biophys. Res. Commun. 128, 1079-1086 (1985).

Higuchi et al., Biotechnology 10:413-417 1992.

Hillenkamp et al. “Matrix Assisted UV-Laser Desorption/Ionization: A New Approach to Mass Spectrometry of Large Biomolecules,” Biological Mass Spectrometry, Burlingame and McCloskey, editors, Elsevier Science Publishers, Amsterdam, pp. 49-60, 1990.

Hofmann, R. M., and Pickart, C. M. Cell 96, 645-653, 1999.

Hopp et al., BioTechnology 6:1205-1210, 1988.

Hsu, H., Shu, H. -B., Pan, M. -P., and Goeddel, D. V. Cell 84, 299-308, 1996.

Hu et al., J. Biol. Chem. 269, 30069-30072, 1994.

Ishida et al. J. Biol. Chem. 271, 28745-28748, 1996.

Jacobson et al., Anal. Chem., 66:1107-1113, 1994.

Japanese Patent No. 59-131909

Joyce, “RNA evolution and the origins of life,” Nature, 338:217-244, 1989.

Kaneda et al., “Increased expression of DNA cointroduced with nuclear protein in adult rat liver,” Science, 243:375-378, 1989.

Kato et al, J. Biol. Chem., 266:3361-3364, 1991

Kim and Chech, “Three-dimensional model of the active site of the self-splicing rRNA precursor of Tetrahymena,” Proc. Nat'l Acad. Sci. USA, 84(24):8788-8792, 1987.

Koster et al., Biomedical Environmental Mass Spectrometry 14, 111-116, 1987.

Kreis et al., EMBO. J 5:931-941, 1986.

Laney, J. D., and Hochstrasser, M. Cell 97, 427-430, 1999.

Lee et al., Proc. Nat'l Acad. Sci. USA 93, 9699-9703, 1996.

Lee, F. S., Peters, R. T., Dang, L. C., and Maniatis, T. Proc. Nat'l Acad. Sci. USA. 95, 9319-9324, 1998.

Lee, F. S., Hagler, J., Chen, Z., and Maniatis, T. Cell 88, 213-222, 1997.

Lee et al., Nuc. Acids Res. 21:3761-3766, 1993.

Liang et al., Arch. Biochem. Biophys. 329:208-214, 1996.

Lieber and Strauss, Mol Cell Biol., 15(1):540-51, 1995.

Lim et al., J. Infect. Dis. 162:1263-1269, 1990.

Lomaga, M. A., et al., Genes Dev. 13, 1015-1024, 1999.

Lorick, K. L., Jensen, J. P., Fang, S., Ong, A. M., Hatakeyama, S., and Weissman, A. M. Proc. Nat'l Acad. Sci. USA 96,11364-11369, 1999.

MacArthur et al., J. Virol. 52:483-491, 1984.

Manz et al., J. Chromatog. 593:253-258, 1992.

Michel and Westhof, “Modelling of the three-dimensional architecture of group I catalytic introns based on comparative sequence analysis,” J. Mol Biol., 216:585-610, 1990.

Mosialos et al., Cell 80, 389-399,1995.

Naito, A., Azuma, S., Tanaka, S., Miyazaki, T., Takaki, S., Takatsu, K., Nakao, K., Nakamura, K., Katsuki, M., Yamamoto, T., and Inoue, J. Genes Cells 4, 353-362, 1999.

Nakano et al. J. Biol. Chem. 271, 14661-14664, 1996.

Nicolau and Sene, “Liposome-mediated DNA transfer in eukaryotic cells. Dependence of the transfer efficiency upon the type of liposomes used and the host cell cycle stage,” Biochim. Biophys. Acta, 721:185-190, 1982.

Nicolau, Legrand, Grosse, “Liposomes as carriers for in vivo gene transfer and expression,” Methods Enzymol., 149:157-176, 1987.

Niman et al., Proc. Nat'l Acad. Sci. USA 80:4949-4953, 1983.

Ninomiya-Tsuji et al., Nature 398, 252-256, 1999.

Nonisotopic DNA Probe Techniques, Academic Press, Inc., pgs. 311-352, 1992.

Oláh et al., Anal. Biochem. 221:94-102, 1994.

P. F. Crain, “Mass Spectrometric Techniques in Nucleic Acid Research,” Mass Spectrometry Reviews 9, 505-554, 1990.

Palombella, V. J., Rando, O. J., Goldberg, A. L., and Maniatis, T., Cell 78, 773-785, 1994.

Pease et al., Proc. Natl. Acad. Sci. USA, 91:5022-5026, 1994.

Perales, Ferkol, Beegen, Ratnoff, Hanson, “Gene transfer in vivo: sustained expression and regulation of genes introduced into the liver by receptor-targeted uptake,” Proc. Nat'l Acad. Sci. 91:4086-4090, 1994.

Perriman, Delves, Gerlach, “Extended target-site specificity for a hammerhead ribozyme,” Gene 113(2):157-163, 1992.

Pickart, C. M. FASEB J. 11, 1055-1066, 1997.

Potter, Weir, Leder, “Enhancer-dependent expression of human kappa immunoglobulin genes introduced into mouse pre-B lymphocytes by electroporation,” Proc. Nat'l Acad. Sci. USA, 81:7161-7165, 1984.

Prody et al., J Neurosci Res. 16(1):25-35, 1986.

Raingeaud et al. Mol. Cell. Biol. 16, 1247-55, 1996.

Reinhold-Hurek and Shub, “Self-splicing introns in tRNA genes of widely divergent bacteria,” Nature, 357:173-176, 1992.

Rippe, Brenner and Leffert, “DNA-mediated gene transfer into adult rat hepatocytes in primary culture,” Mol. Cell Biol., 10:689-695, 1990.

Rothe, M., Sarma, V., Dixit, V. M., and Goeddel, D. V. Science 269, 1424-1427 1995.

Rothe, M., Wong, S. C., Henzel, W. J., and Goeddel, D. V. Cell 78, 681-692, 1994.

Rothofsky, M. L., and Lin, S. L. Gene 195, 141-149, 1997.

Sambrook et al., “Molecular Cloning,” A Laboratory Manual, 2d Ed., Cold Spring Harbor Laboratory Press, New York, 13.7-13.9:1989.

Sancho et al., Mol. Cell. Biol. 18, 576-589, 1998.

Sato, T., Irie, S., and Reed, J. C. FEBS Lett. 358, 113-118, 1995.

Sato et al., Ann. NY Acad. Sci., 417:213-223, 1983.

Sanz, L., Diaz-Meco, M. T., Nakano, H., Moscat, J. EMBO J. 19, 1576-1586, 2000.

Schram, “Mass Spectrometry of Nucleic Acid Components, Biomedical Applications of Mass Spectrometry” Meth. Biochem. Anal. 34, 203-287, 1990.

Sen, R., and Baltimore, D. Cell 47, 921-928, 1986.

Shinkura, R., Kitada, K., Matsuda, F., Tashiro, K., Ikuta, K., Suzuki, M., Kogishi, K., Serikawa, T., and Honjo, T. Nat. Genet. 22, 74-77,1999.

Shoemaker et al., Nature Genetics 14:450-456, 1996.

Smith et al., Anal. Chem. 62, 882-89, 1990.

Smith, C. A., Farrah, T., and Goodwin, R. G. Cell 76, 959-962, 1994.

Song, H. Y., and Donner, D. B. Biochem. J 809, 825-829, 1995

Spencer, E., Jiang, J., Chen, Z. J. Genes Dev. 13, 284-294, 1999.

Symons, “Small catalytic RNAs.” Annu. Rev. Biochem., 61:641-671, 1992.

Takaesu, G. et al. Mol. Cell 5, 649-658, 2000.

Thompson et al., “Decreased expression of BRCA1 accelerates growth and is often present during sporadic breast cancer progression,” Nature Genet., 9:444-450, 1995.

Thompson et al., “Ribozymes in gene therapy.” Nature Medicine, 1:277-278, 1995.

Tsuda et al., Anal. Chem., 62:2149-2152, 1990.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

U.S. Pat. No. 5,547,861

U.S. Pat. No. 3,817,837

U.S. Pat. No. 3,850,752

U.S. Pat. No. 3,939,350

U.S. Pat. No. 3,996,345

U.S. Pat. No. 4,277,437

U.S. Pat. No. 4,275,149

U.S. Pat. No. 4,366,241

U.S. Pat. No. 5,994,624

U.S. Pat. No. 5,981,274

U.S. Pat. No. 5,945,100

U.S. Pat. No. 5,780,448

U.S. Pat. No. 5,736,524

U.S. Pat. No. 5,702,932

U.S. Pat. No. 5,656,610

U.S. Pat. No. 5,589,466

U.S. Pat. No. 5,580,859

U.S. Pat. No. 5,789,215

U.S. Pat. No. 5,384,253

U.S. Pat. No. 5,354,855

U.S. Pat. No. 5,908,845

U.S. Pat. No. 5,786,461

U.S. Pat. No. 5,891,625

U.S. Pat. No. 5,773,571

U.S. Pat. No. 5,766,855

U.S. Pat. No. 5,736,336

U.S. Pat. No. 5,719,262

U.S. Pat. No. 5,714,331

U.S. Pat. No. 5,539,082

U.S. Pat. No. 5,891,625

U.S. Pat. No. 5,786,461

U.S. Pat. No. 5,168,053

U.S. Pat. No. 5,624,824

Wagner et al., Proc. Nat'l Acad. Sci. USA, 87(9):3410-3414, 1990.

Wagner et al., Science, 260:1510-1513, 1990.

Wang et al., Gene 169:53-58, 1996.

Williams et al., “Volatilization of High Molecular Weight DNA by Pulsed Laser Ablation of Frozen Aqueous Solutions,” Science 246, 1585-87, 1989.

WO 84/03564

WO 90/14148

WO 92/20702

WO 96/21144

Woolley and Mathies, Proc Nat'l Acad Sci USA, 91:11348-52, 1994.

Wong, Nicolau, Hofschneider, “Appearance of β-lactamase activity in animal cells upon liposome mediated gene transfer,” Gene, 10:87-94, 1980.

Wu & Wu, J. Biol. Chem., 262:4429-4432, 1987.

Wu and Wu, Biochemistry, 27:887-892, 1988.

Wu and Wu, Adv. Drug Delivery Rev., 12:159-167, 1993.

You, J., Cohen, R. E., and Pickart, C. M., Biotechniques 27, 950-954, 1999.

Yuan and Altman, “Selection of guide sequences that direct efficient cleavage of mRNA by human ribonuclease P,” Science, 263:1269-1273, 1994.

Yuan, Hwang, Altman, “Targeted cleavage of mRNA by human RNase P,” Proc. Nat'l Acad. Sci. USA, 89:8006-8010, 1992.

What is claimed is:

1. A method of screening for modulators of IKK and JNK activation comprising:
(a) providing a Ubc13/Uev1A complex and TRAF6 or TRAF2;
(b) contacting said complex and TRAF6 or TRAF2 with a candidate modulator substance in the presence of E1, a plurality of ubiquitin molecules and ATP;
(c) determining the formation of free polyubiquitin chains,
wherein a change in poly-ubiquitin formation in the presence of said candidate modulator, as compared with poly-ubiquitin formation in the absence of said candidate modulator, indicates that said candidate modulator is an modulator of IKK and JNK activation.

2. The method of claim 1, further comprising measuring poly-ubiquitin formation in the absence of said candidate modulator.

3. The method of claim 1, wherein said candidate modulator is a protein or peptide.

4. The method of claim 1, wherein said candidate modulator is an expression construct.

5. The method of claim 1, wherein candidate modulator is an organic small molecule.

6. The method of claim 1, wherein said candidate modulator is an inhibitor.

7. The method of claim 1, wherein said candidate modulator is a stimulator.

8. The method of claim 1, wherein candidate modulator is an inorganic small molecule.

9. The method of claim 1, wherein candidate modulator is a DNA oligonucleotide or its analogue.

10. The method of claim 1, wherein candidate modulator is an RNA oligonucleotide or its analogue.

11. The method of claim 2, further comprising measuring poly-ubiquitin formation by immunodetection.

12. The method of claim 11, wherein immunodetection comprises detecting ubiquitin fused to an immunodetectable marker.

13. The method of claim 13, wherein said immunodetectable marker is myc or His6X.

14. The method of claim 11, wherein the format of the immunodetection is ELISA.

15. The method of claim 2, wherein the plurality of ubiquitin molecules comprise a single lysine residue at position 63.

16. A method of screening for modulators of IKK and JNK activation comprising:
(a) providing a Ubc13/Uev1A complex, TRAF6 or TRAF2;
(b) contacting TAB1/TAB2/TAK1 complex, Ubc13/Uev1A complex, TRAF6 or TRAF2 with a candidate modulator substance in the presence of E1, a plurality of ubiquitin molecules and ATP;
(c) determining the polyubiquitination of TRAF6 or TRAF2, wherein a change in the phosphorylation state of IKK or MKK in the presence of said candidate modulator, as compared with the phosphorylation state of IKK or MKK in the absence of said candidate modulator, indicates that said candidate modulator is an modulator of IKK and JNK activation.

17. The method of claim 16, further comprising measuring the polyubiquitination state of TRAF6 or TRAF2 in the absence of said candidate modulator.

18. The method of claim 16, wherein said candidate modulator is a protein or peptide.

19. The method of claim 16, wherein said candidate modulator is an expression construct.

20. The method of claim 16, wherein candidate modulator is an organic small molecule.

21. The method of claim 16, wherein said candidate modulator is an inhibitor.

22. The method of claim 16, wherein said candidate modulator is a stimulator.
The method of claim 16, wherein candidate modulator is an inorganic small molecule.

23. The method of claim 16, wherein candidate modulator is a DNA oligonucleotide or its analogue.

24. The method of claim 16, wherein candidate modulator is an RNA oligonucleotide or its analogue.

25. The method of claim 17, further comprising measuring poly-ubiquitin state of TRAF6 or TRAF2 by immunodetection.

26. The method of claim 25, wherein immunodetection comprises detecting TRAF2, TRAF6 or ubiquitin fused to an immunodetectable marker.

27. The method of claim 26, wherein said ubiquitin molecule comprises a single lysine residue at position 63.

28. The method of claim 26, wherein said immunodetectable marker is 6HisX or myc.

29. The method of claim 25, wherein the format of said immunodetection is ELISA.

30. A method of screening for modulators of IKK and JNK activation comprising:
(a) providing a TRAF2 or a TRAF6;
(b) contacting TRAF2 or TRAF6 with a candidate modulator substance in the presence of E1, Ubc13/Uev1A, a plurality of ubiquitin molecules and ATP;
(c) determining the ubiquitin ligase activity of TRAF2 or TRAF6,
wherein a change in the enzyme activity of TRAF2 or TRAF6 in the presence of said candidate modulator, as compared with the activity of TRAF2 or TRAF6 in the absence of said candidate modulator, indicates that said candidate modulator is an modulator of IKK and JNK activation.

31. The method of claim 30, further comprising measuring TRAF2 or TRAF6 activity in the absence of said candidate modulator.

32. The method of claim 30, wherein said candidate modulator is a protein or peptide.

33. The method of claim 30, wherein said candidate modulator is an expression construct.

34. The method of claim 30, wherein candidate modulator is an organic or inorganic small molecule.

35. The method of claim 30, wherein said candidate modulator is an inhibitor.

36. The method of claim 30, wherein said candidate modulator is a stimulator.

37. The method of claim 30, wherein candidate modulator is a DNA oligonucleotide or its analogue.

38. The method of claim 30, wherein candidate modulator is an RNA oligonucleotide or its analogue.

39. The method of claim 30, further comprising measuring poly-ubiquitin formation by immunodetection.

40. The method of claim 39, wherein immunodetection comprises detecting ubiquitin fused to an immunodetectable marker.

41. The method of claim 40, wherein said immunodetectable marker is myc or His6X.

42. A method of screening for modulators of IKK and JNK activation comprising:
(a) providing TAK1, TAB 1, TRAF6 and TAB2;
(b) contacting TAK1, TAB 1, TRAF6 and TAB2 with a candidate modulator substance in the presence of E1, Ubc13/Uev1A, a plurality of ubiquitin molecules and ATP;
(c) determining the kinase activity of TAK 1,
wherein a change in the kinase activity of TAK1 in the presence of said candidate modulator, as compared with the kinase activity of TAK1 in the absence of said candidate modulator, indicates that said candidate modulator is an modulator of IKK and JNK activation.

43. The method of claim 42, further comprising measuring TAK1 activity in the absence of said candidate modulator.

44. The method of claim 42, wherein said candidate modulator is a protein or peptide.

45. The method of claim 42, wherein said candidate modulator is an expression construct.

46. The method of claim 42, wherein candidate modulator is an organic small molecule.

47. The method of claim 42, wherein said candidate modulator is an inhibitor.

48. The method of claim 42, wherein said candidate modulator is a stimulator.
The method of claim 42, wherein candidate modulator is an inorganic small molecule.

49. The method of claim 42, wherein candidate modulator is a DNA oligonucleotide or its analogue.

50. The method of claim 42, wherein candidate modulator is an RNA oligonucleotide or its analogue.

51. The method of claim 42, wherein TAK1 kinase activity is measured by determining the phosophorylation state of IκBα incubated with said TAK1.

52. The method of claim 51, wherein said phosphorylation state is determined with an antibody that binds selectively to phosphorylated IκBα.

53. The method of claim 52, wherein the format of the assay is ELISA.

54. A method of screening for modulators of IKK and JNK activation comprising:
(a) providing TAK1, TAB 1, TAB2, TRAF6 and IKK complex;
(b) contacting TAK1, TAB1, TAB2, TRAF6 and IKK complex with a candidate modulator substance in the presence of E1, Ubc13/Uev1A, a plurality of ubiquitin molecules and ATP;
(c) determining the phosphorylation state of IκB and MKK,
wherein a change in the phosphorylation state of IκB and MKK in the presence of said candidate modulator, as compared with the phosphorylation state of IκB and MKK in the absence of said candidate modulator, indicates that said candidate modulator is an modulator of IKK and JNK activation.

55. The method of claim 54, further comprising measuring the phosphorylation of IκBα or MKK in the absence of said candidate modulator.

56. The method of claim 54, wherein said candidate modulator is a protein or peptide.

57. The method of claim 54, wherein said candidate modulator is an expression construct.

58. The method of claim 54, wherein candidate modulator is an organic small molecule.

59. The method of claim 54, wherein said candidate modulator is an inhibitor.

60. The method of claim 54, wherein said candidate modulator is a stimulator.
The method of claim 54, wherein candidate modulator is an inorganic small molecule.

61. The method of claim 54, wherein candidate modulator is a DNA oligonucleotide or its analogue.

62. The method of claim 54, wherein candidate modulator is an RNA oligonucleotide or its analogue.

63. The method of claim 54, wherein said phosphorylation state of IκBαis determined with an antibody that binds selectively to phosphorylated IκBα.

64. The method of claim 54, wherein said phosphorylation state of MKK is determined with an antibody that binds selectively to phosphorylated MKK.

65. The method of claim 54, wherein the format of the assay is ELISA.

66. The method of claim 55, wherein the format of the assay is ELISA.
